(19) United States
(12) Patent Application Publication
(10) Pub. No.: US 2026/0251537 A1
(43) Pub. Date: Aug. 27, 2026

(54) GAS LEAK DETECTION IN OUTSIDE ENVIRONMENTS

(71) Applicant: Chevron U.S.A. Inc., San Ramon, CA (US)

(72) Inventor: Daniel Concannon Segal, Castro Valley, CA (US)

(21) Appl. No.: 19/060,131

(22) Filed: Feb. 21, 2025

(51) Int. Cl. G01N 1/22; G01M 3/04; G01N 1/26; G01N 33/00

(52) U.S. Cl. CPC G01N 1/2202; G01M 3/04; G01N 1/26; G01N 33/0014; G01N 33/004; G01N 33/005

(57) ABSTRACT

A method for detecting a leak of a gas from a storage vessel in an outside environment may include obtaining at a location in the outside environment an original sample of the outside environment at the location; processing the original sample to generate a processed sample by removing a naturally-occurring quantity of the gas from the original sample; obtaining a measurement of a parameter associated with the gas in the processed sample; comparing the measurement to a range of acceptable values; determining that the measurement falls outside the range of acceptable values; identifying, based on the location and based on determining that the measurement falls outside the range of acceptable values, a characteristic of the leak in the storage vessel; and communicating discovery of the leak in the storage vessel at the location.

### TECHNICAL FIELD

The present application is related to detecting gas leaks and, more particularly, to detecting gas leaks in outdoor or open air environments.

### BACKGROUND

A number of different gases (e.g., dihydrogen (H₂), carbon dioxide (CO₂)) may be stored for different reasons (e.g., for use in a process, for sequestration in the mitigation of greenhouse gas emissions) and in any of a number of different vessels (e.g., a tank, a container, a layer of a subterranean formation). In some cases, if leaks develop in these storage vessels, the gas contained therein may be released into the surrounding environment. Some of these gases (or at least some of their chemical elements and/or variations of the gases) may already exist naturally in these surrounding environments, and so detecting leaks in the storage vessels that store these gases may be difficult to detect.

### SUMMARY

In general, in one aspect, the disclosure relates to a method for detecting a leak of a gas from a storage vessel in an outside environment. The method may include obtaining, by a sample collector on a mobile vehicle at a location in the outside environment, an original sample of the outside environment at the location. The method may also include processing, in real time by a processing apparatus on the mobile vehicle, the original sample to generate a processed sample by removing a naturally-occurring quantity of the gas from the original sample. The method may further include obtaining, from a sensor device on the mobile vehicle, a measurement of a parameter associated with the gas in the processed sample. The method may also include comparing, in real time using an analytic system on the mobile vehicle, the measurement to a range of acceptable values. The method may further include determining, in real time by the analytic system, that the measurement falls outside the range of acceptable values. The method may also include identifying, in real time based on the location and based on determining that the measurement falls outside the range of acceptable values, a characteristic of the leak in the storage vessel. The method may further include reporting the leak in the storage vessel at the location in real time.

In another aspect, the disclosure relates to a mobile vehicle used to detect a leak of a gas from a storage vessel in an outside environment. The mobile vehicle may include a sample collector configured to collect a plurality of original samples within the outside environment, where each of the plurality of original samples comprises the gas. The mobile vehicle may also include a processing apparatus configured to remove a naturally-occurring quantity of the gas from the plurality of samples to generate a plurality of processed samples. The mobile vehicle may further include a sensor device configured to measure an amount of the gas in each of the plurality of processed samples. The mobile vehicle may also include an analytic system configured to compare, in real time, each measurement to a range of acceptable values. The analytic system may also be configured to determine, in real time, that at least one measurement falls outside the range of acceptable values. The analytic system may further be configured to identify, in real time based and based on determining that the at least one measurement falls outside the range of acceptable values, a source of the leak in the storage vessel.

In yet another aspect, the disclosure relates to a computer-implemented method for detecting a leak of a gas from a storage vessel in an outside environment using a mobile vehicle. The computer-implemented method may include facilitate positioning the mobile vehicle at a location in the outside environment. The computer-implemented method may also include facilitate obtaining, by a sample collector on the mobile vehicle at the location in the outside environment, an original sample of the outside environment at the location. The computer-implemented method may further include facilitate processing, in real time by a processing apparatus on the mobile vehicle, the original sample to generate a processed sample by removing a naturally-occurring quantity of the gas from the original sample. The computer-implemented method may also include facilitate obtaining, from a sensor device on the mobile vehicle, a measurement of a parameter associated with the gas in the processed sample. The computer-implemented method may further include facilitate comparing, in real time using an analytic system on the mobile vehicle, the measurement to a range of acceptable values. The computer-implemented method may also include facilitate determining, in real time by the analytic system, that the measurement falls outside the range of acceptable values. The computer-implemented method may further include facilitate identifying, in real time based on the location and based on determining that the measurement falls outside the range of acceptable values, a characteristic of the leak in the storage vessel. The computer-implemented method may also include facilitate reporting the leak in the storage vessel at the location in real time

These and other aspects, objects, features, and embodiments will be apparent from the following description and the appended claims.

### BRIEF DESCRIPTION OF THE DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, the same reference numerals used in different figures may designate like or corresponding but not necessarily identical elements.

FIG. 1 shows a field system in which example embodiments may be used.

FIG. 2 shows another field system in which example embodiments may be used.

FIG. 3 shows yet another field system in which example embodiments may be used.

FIG. 4 shows a diagram of a system for detecting a leak of a gas from a storage vessel in an outside environment using a mobile vehicle according to certain example embodiments.

FIG. 5 shows a system diagram of a controller of an analysis system on a mobile vehicle according to certain example embodiments.

FIG. 6 shows a computing device in accordance with certain example embodiments.

FIG. 7 shows a flowchart of a method for detecting a leak of a gas from a storage vessel in an outside environment using a mobile vehicle according to certain example embodiments.

FIG. 8 shows a top view of a system in which an example mobile vehicle tests for leaks around 12 storage vessels in accordance with certain example embodiments.

FIG. 9 shows a top view of another system in which an example mobile vehicle tests for leaks around 4 storage vessels in accordance with certain example embodiments.

FIG. 10 shows a system that includes an example mobile vehicle according to certain example embodiments.

### DETAILED DESCRIPTION

The example embodiments discussed herein are directed to systems, apparatus, methods, and devices for detecting a leak of a gas from a storage vessel in an outside environment using a mobile vehicle. In some cases, use of example embodiments may allow for more timely and efficient detection of gas leaks. Further, example embodiments may allow for an estimate of the extent and location of a gas leak.

The use of the terms “about”, “approximately”, and similar terms applies to all numeric values, whether or not explicitly indicated. These terms generally refer to a range of numbers that one of ordinary skill in the art would consider as a reasonable amount of deviation to the recited numeric values (i.e., having the equivalent function or result). For example, this term may be construed as including a deviation of ±10 percent of the given numeric value provided such a deviation does not alter the end function or result of the value. Therefore, a value of about 1% may be construed to be a range from 0.9% to 1.1%. Furthermore, a range may be construed to include the start and the end of the range. For example, a range of 10% to 20% (i.e., range of 10%-20%) includes 10% and also includes 20%, and includes percentages in between 10% and 20%, unless explicitly stated otherwise herein. Similarly, a range of between 10% and 20% (i.e., range between 10%-20%) includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if an item is described herein as including a component of type A, a component of type B, a component of type C, or any combination thereof, it is understood that this phrase describes all of the various individual and collective combinations and permutations of these components. For example, in some embodiments, the item described by this phrase could include only a component of type A.

In some embodiments, the item described by this phrase could include only a component of type B. In some embodiments, the item described by this phrase could include only a component of type C. In some embodiments, the item described by this phrase could include a component of type A and a component of type B. In some embodiments, the item described by this phrase could include a component of type A and a component of type C. In some embodiments, the item described by this phrase could include a component of type B and a component of type C. In some embodiments, the item described by this phrase could include a component of type A, a component of type B, and a component of type C.

In some embodiments, the item described by this phrase could include two or more components of type A (e.g., A1 and A2). In some embodiments, the item described by this phrase could include two or more components of type B (e.g., B1 and B2). In some embodiments, the item described by this phrase could include two or more components of type C (e.g., C1 and C2). In some embodiments, the item described by this phrase could include two or more of a first component (e.g., two or more components of type A (A1 and A2)), optionally one or more of a second component (e.g., optionally one or more components of type B), and optionally one or more of a third component (e.g., optionally one or more components of type C).

In some embodiments, the item described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the item described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

If a component of a figure is described but not expressly shown or labeled in that figure, the label used for a corresponding component in another figure may be inferred to that component. Conversely, if a component in a figure is labeled but is not described, the description for such component may be substantially the same as the description for the corresponding component in another figure. The numbering scheme for the various components in the figures herein is such that each component is a three-digit number or a four-digit number, and corresponding components in other figures have the identical last two digits. For any figure shown and described herein, one or more of the components may be omitted, added, repeated, and/or substituted. Accordingly, embodiments shown in a particular figure should not be considered limited to the specific arrangements of components shown in such figure.

Further, a statement that a particular embodiment (e.g., as shown in a figure herein) does not have a particular feature or component does not mean, unless expressly stated, that such embodiment is not capable of having such feature or component. For example, for purposes of present or future claims herein, a feature or component that is described as not being included in an example embodiment shown in one or more particular drawings is capable of being included in one or more claims that correspond to such one or more particular drawings herein.

Example embodiments of detecting a leak of a gas from a storage vessel in an outside environment using a mobile vehicle will be described more fully hereinafter with reference to the accompanying drawings, in which example embodiments of detecting a leak of a gas from a storage vessel in an outside environment using a mobile vehicle are shown. Detecting a leak of a gas from a storage vessel in an outside environment using a mobile vehicle may, however, be embodied in many different forms and should not be construed as limited to the example embodiments set forth herein. Rather, these example embodiments are provided so that this disclosure will be thorough and complete, and will fully convey the scope of detecting a leak of a gas from a storage vessel in an outside environment using a mobile vehicle to those of ordinary skill in the art. Like, but not necessarily the same, elements (also sometimes called components) in the various figures are denoted by like reference numerals for consistency.

Terms such as “first”, “second”, “primary,” “secondary,” “above”, “below”, “inner”, “outer”, “distal”, “proximal”, “end”, “top”, “bottom”, “upper”, “lower”, “side”, “left”, “right”, “front”, “rear”, and “within”, when present, are used merely to distinguish one component (or part of a component or state of a component) from another. This list of terms is not exclusive. Such terms are not meant to denote a preference or a particular orientation, and they are not meant to limit embodiments of detecting a leak of a gas from a storage vessel in an outside environment using a mobile vehicle. In the following detailed description of the example embodiments, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

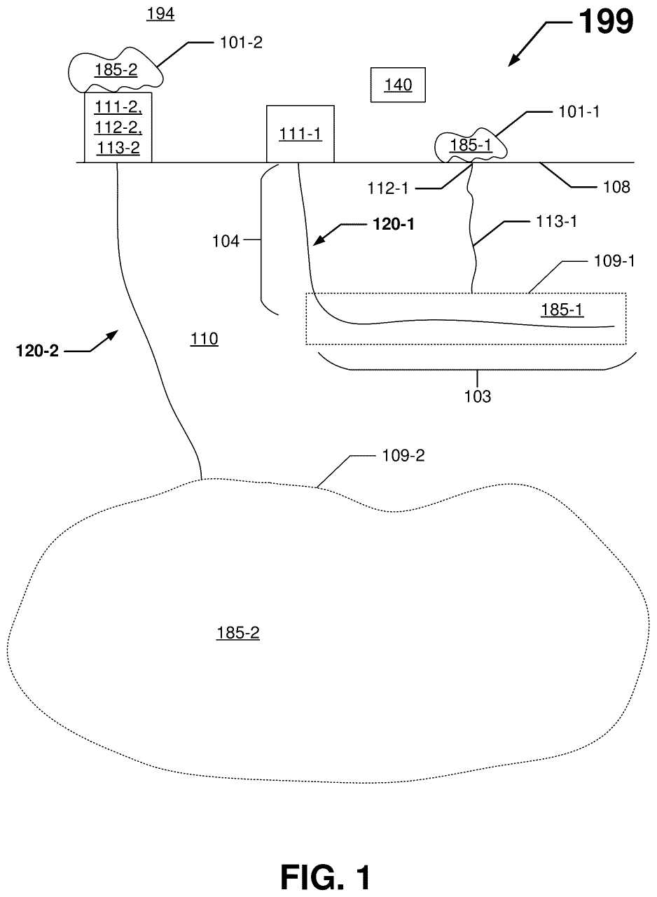

FIG. 1 shows a schematic diagram of a land-based field system 199 with which example embodiments may be used. The field system 199 of FIG. 1 includes two wellbores 120 (wellbore 120-1 and wellbore 120-2) that are drilled into the same subterranean formation 110. Both wellbores 120 in this case are used as injection wells for storage. Specifically, at the end of wellbore 120-1 is a storage vessel 109-1 formed by part of the subterranean formation 110. A gas 185-1 is stored within the storage vessel 109-1. Similarly, at the end of wellbore 120-2 is a storage vessel 109-2 formed by part of the subterranean formation 110. A gas 185-2 is stored within the storage vessel 109-2.

The subterranean formation 110 refers to practically any volume under the ground 108. Each subsurface volume of interest (also called a storage vessel 109 herein) may have a variety of characteristics, including but not limited to petrophysical rock properties, reservoir fluid properties, reservoir conditions, or any combination thereof. For example, each subsurface storage vessel 109 may be associated with one or more of: temperature, porosity, salinity, permeability, water composition, mineralogy, reservoir location, pressure, etc. The terms “formation”, “subsurface formation”, “hydrocarbon-bearing formation”, “reservoir”, “subsurface reservoir”, “subsurface area of interest”, “subsurface region of interest”, “subsurface volume of interest”, and the like may be used synonymously with subterranean formation 110.

Each wellbore 120 (also sometimes called a well) refers to a single hole, usually cylindrical, that is drilled into the subterranean formation 110. A wellbore 120 may be drilled in one or more directions. For example, a well or a wellbore may include a vertical well, a horizontal well, a deviated well, and/or other type of well. Multiple (e.g., tens to hundreds) of wellbores 120 are sometimes drilled in a subterranean formation 110 depending on the desired outcome. A wellbore 120 may be drilled into the subterranean formation 110 using practically any drilling technique and equipment known in the art, such as geosteering, directional drilling, etc.

Drilling a wellbore 120 may include using a tool, such as a drilling tool that includes a drill bit and a drill string. Drilling fluid, such as drilling mud, may be used while drilling in order to cool the drill tool and remove cuttings. Other tools may also be used while drilling or after drilling, such as measurement-while-drilling (MWD) tools, seismic-while-drilling tools, wireline tools, logging-while-drilling (LWD) tools, or other downhole tools. After drilling to a predetermined depth, the drill string and the drill bit may be removed, and then casing, the tubing, and/or other equipment may be installed according to the design of the wellbore 120. The equipment to be used in drilling the wellbore 120 may be dependent on the design of the wellbore 120, the layers of the subterranean formation 110, and/or other factors.

In this example, wellbore 120-1 is configured as a horizontal well, where the wellbore 120-1 has at least one substantially vertical section 104 and at least one substantially horizontal section 103. A horizontal section 103 is located toward the distal end of the wellbore 120-1 and may be located in a layer of the subterranean formation that may be considered unconventional (e.g., shale, tight formations). The layer of the subterranean formation 110 in which the substantially horizontal section 103 is positioned forms a storage vessel 109-1 in which a gas 185-1 may be stored. In horizontal wells, fractures may be induced along some or all of the substantially horizontal section 103 to allow for larger (e.g., in terms of number, in terms of length, in terms of width) flow paths between the wellbore 120-1 and the layer of the subterranean formation 110, which increases the size of the storage vessel 109-1.

By contrast, wellbore 120-2 is configured as a vertical well, where the wellbore 120-2 does not have any substantially horizontal sections. The layer of the subterranean formation 110 in which the distal end of the wellbore 120-2 is positioned forms a storage vessel 109-2 in which a gas 185-2 may be stored. In vertical wells, fracturing may not be needed to increase the size of the storage vessel 109-1 because the layer of the subterranean formation 110 in which the distal end of the wellbore 120-2 is positioned is more conventional (e.g., sandstone, limestone) and/or is a salt dome or similar type of vacuous space suitable for storage.

In any case, a wellbore 120 used for storage of a gas 185 may be abandoned (e.g., as from production of oil, gas, water, etc.) or drilled specifically as an injection well. Each wellbore 120 has field equipment 111 located on or near the ground 108 at the entry point of the wellbore 120. If the wellbore 120 is being used for active injection of a gas 185 into the storage vessel 109 in the subterranean formation 110, then the field equipment 111 may include pumps, motors, compressors, and other equipment that allow the gas 185 to be injected into the wellbore 120. If the wellbore 120 is no longer being used for active injection of a gas 185 into the storage vessel 109 in the subterranean formation 110, then the field equipment 111 may include a cap and/or similar equipment to keep the wellbore 120 sealed so that the gas 185 in the storage vessel 109 is unable to enter the outside environment 194 through the wellbore 120. In this case, field equipment 111-1 is positioned at the entry point of wellbore 120-1, and field equipment 111-2 is positioned at the entry point of wellbore 120-2. For purposes herein, the field equipment 111 is considered part of the storage vessel 109. In this case, the field equipment 111-1 is considered part of the storage vessel 109-1, and the field equipment 111-2 is considered part of the storage vessel 109-2.

In some cases, however, the field equipment 111 may have a failure or malfunction that allows gas 185 stored in a storage vessel 109 to escape into the outside environment 194 through the wellbore 120. For example, in this case, some of gas 185-2 stored in storage vessel 109-2 in the subterranean formation is able to escape into the outside environment 194 through the wellbore 120-2 because of a failure or malfunction in the field equipment 111-2. As a result, there is a failure 113-2 of the field equipment 111-2, which results in a leak point 112-2 (also sometimes called a source of the leak 101-2) through which a leak 101-2 (sometimes referred to as a plume herein) of the gas 185-2 escapes into the outside environment 194.

In other cases, rather than the field equipment 111 failing, the storage vessel 109 itself (in this case, in the form of the subterranean formation 110) may have a failure that allows a gas 185 to escape from the storage vessel 109 into the outside environment 194. For example, in this case, there is a failure 113-1 in the form of one or more fractures (e.g., naturally occurring fractures) that span from the storage vessel 109-1 to the ground 108, which results in a leak point 112-1 (also sometimes called a source of the leak 101-1) at the ground 108 through which a leak 101-1 of the gas 185-1 escapes into the outside environment 194.

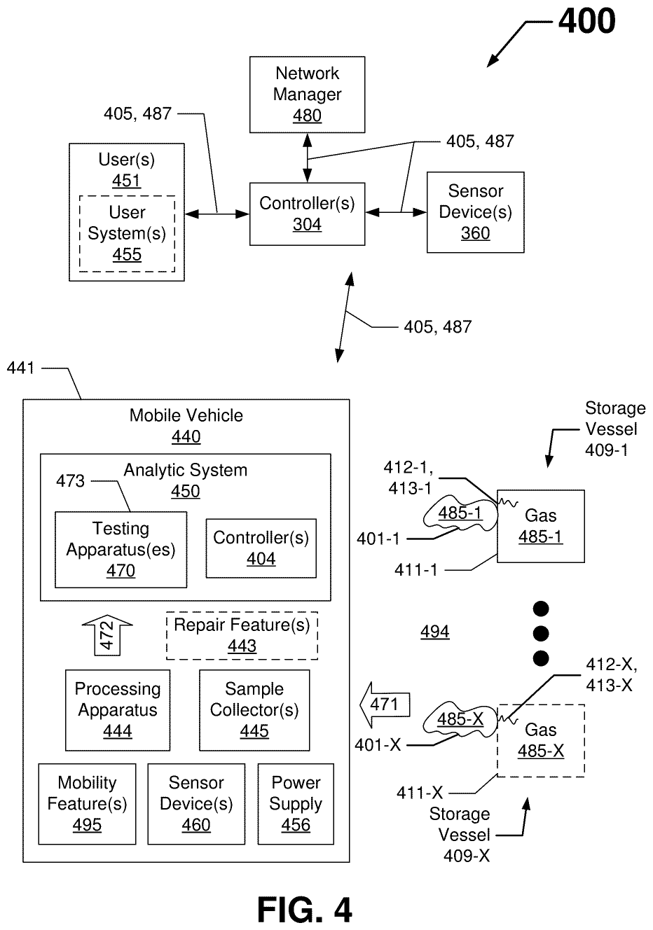

To detect the leak 101-1 of the gas 185-1 that escapes from the storage vessel 109-1 through the leak point 112-1 to the outside environment 194, and to detect the leak 101-2 of the gas 185-2 that escapes from the storage vessel 109-2 through the leak point 112-2 to the outside environment 194, an example mobile vehicle 140 may be used. In this case, the mobile vehicle 140 is capable of traveling above the ground 108 (e.g., in the form of a drone or other unmanned aerial vehicle (UAV)). More details about the mobile vehicle 140 are described below with respect to FIG. 4.

The gas 185-1 that is contained within and escapes from the storage vessel 109-1 may be the same as (e.g., in terms of chemical composition, in terms of purity), or different than, the gas 185-2 that is contained within and escapes from the vessel 109-2. In any case, the example mobile vehicle 140 may be configured to identify (and in some cases quantify and locate) the leak 101-1 of the gas 185-1 from the storage vessels 109-1 and the leak 101-2 of the gas 185-2 from the storage vessel 109-2.

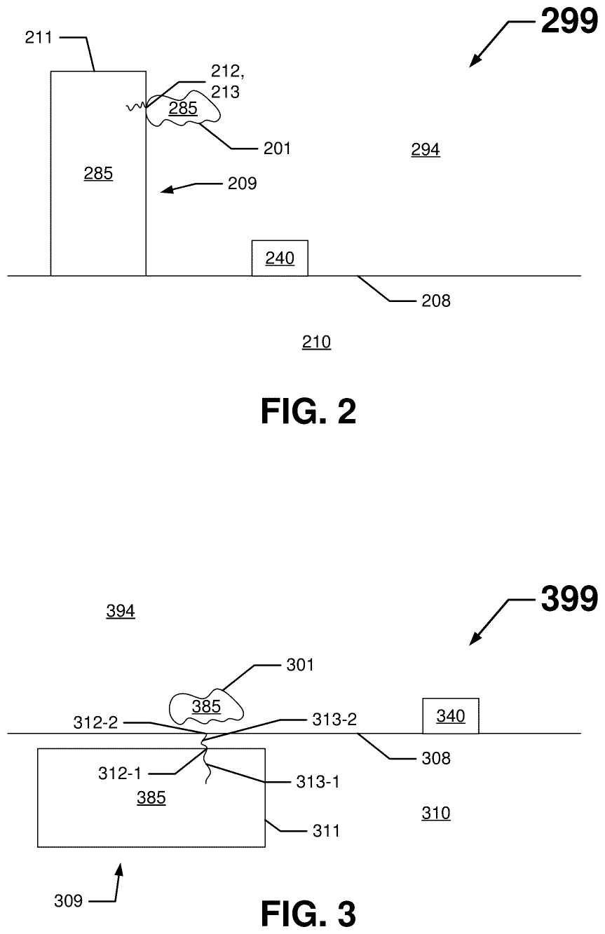

FIG. 2 shows another field system 299 in which example embodiments may be used. Referring to the description above with respect to FIG. 1, the field system 299 of FIG. 2 includes a storage vessel 209 used to store a gas 285 that is mounted on the ground 208 above the subterranean formation 210. In this way, the storage vessel 209 is substantially exposed to the outside environment 294. Examples of the storage vessel 209 in this case may include, but are not limited to, a tank, a drum, and a silo. The storage vessel 209 has field equipment 211 in the form of one or more walls that form the storage vessel 209. In this case, the storage vessel 209 has a failure 213 in the field equipment 211 in the form of a crack in one of its walls, which creates a leak point 212 (also sometimes called a source of the leak 201) at the outer surface of the wall at the failure 213. The failure 213 allows some of the gas 285 in the storage vessel 209 to accumulate as a leak 201 in the outside environment 294 through the leak point 212.

To detect the leak 201 of the gas 285 that escapes from the storage vessel 209 through the leak point 212 to the outside environment 294, an example mobile vehicle 240 may be used. In this case, the mobile vehicle 240 is capable of traveling along the ground 208 (e.g., in the form of a motor vehicle, in the form of a crawler). As mentioned above, more details about the mobile vehicle 240 are described below with respect to FIG. 4.

FIG. 3 shows yet another field system 399 in which example embodiments may be used. Referring to the description above with respect to FIGS. 1 and 2, the field system 399 of FIG. 3 includes a storage vessel 309 used to store a gas 385 that is buried a shallow distance below the ground 308 within the subterranean formation 310. Examples of the storage vessel 309 in this case may include, but are not limited to, a tank, a drum, and a vault. The storage vessel 309 has field equipment 311 in the form of one or more walls that form the storage vessel 309. In this case, the storage vessel 309 has a failure 313-1 in the field equipment 311 in the form of a crack in one of its walls, which allows some of the gas 385 in the storage vessel 309 to escape the storage vessel 309 through a leak point 312-1 (also sometimes called a source of the leak 301) into the subterranean formation 310. When the subterranean formation 310 between the storage vessel 309 and the ground 308 also has a failure 313-2 (e.g., in the form of naturally occurring fractures, in the form of loose and/or porous soil), the failure 313-2 allows some of the gas 385 that escaped the storage vessel 309 into the subterranean formation 310 to accumulate as a leak 301 in the outside environment 394 through the leak point 312-2 at the ground 308.

To detect the leak 301 of the gas 385 that escapes from the storage vessel 309 through the leak point 312-1 in the storage vessel 309 and through the leak point 312-2 at the ground 308 to the outside environment 394, an example mobile vehicle 340 may be used. In this case, the mobile vehicle 340 is capable of traveling along the ground 308 (e.g., in the form of a motor vehicle, in the form of a crawler). As mentioned above, more details about the mobile vehicle 340 are described below with respect to FIG. 4.

FIG. 4 shows a diagram of a system 400 for detecting a leak of a gas 485 from a storage vessel 409 in an outside environment 494 using a mobile vehicle 440 according to certain example embodiments. The system 400 of FIG. 4 includes an example mobile vehicle 440 and one or more storage vessels 409 located in an outside environment 494, one or more controllers 304, one or more sensor devices 360, one or more users 451 (including one or more optional user systems 455), and a network manager 480. The example mobile vehicle 440 in this case includes an analytic system 450 (which includes one or more controllers 404 and one or more testing apparatuses 470), a body 441, one or more mobility features 495, one or more sensor devices 460, one or more sample collectors 445, one or more optional repair features 443, and a processing apparatus 444. Each storage vessel 409 contains a gas 485 that is retained within the vessel by field equipment 411.

The components shown in FIG. 4 are not exhaustive, and in some embodiments, one or more of the components shown in FIG. 4 may not be included in the example system 400. Any component of the system 400 may be discrete or combined with one or more other components of the system 400. Also, one or more components of the system 400 may have different configurations. For example, one or more sensor devices 360 may be disposed within or disposed on other components (e.g., the field equipment 411 of a storage vessel 409, a valve). As another example, a controller 304, rather than being a stand-alone device, may be part of one or more other components (e.g., a storage vessel 409) of the system 400.

Incorporating the description above with respect to FIGS. 1 through 3, the system 400 of FIG. 4 includes any number (e.g., 1, 2, 5, 9, 18, 25, 50, 1000) of storage vessels 409. In this case, there are X storage vessels 409 (storage vessel 409-1 through storage vessel 409-X). Each of the storage vessels 409 of the system 400 may be substantially similar to the vessels discussed above. For example, a storage vessel 409 may be positioned within a subterranean formation (e.g., subterranean formation 110) and accessible via a wellbore (e.g., wellbore 120). As another example, a storage vessel 409 may be a tank, container, vault, barrel, etc. that is located above ground or buried in the ground. Each storage vessel 409 is configured to hold a volume of gas 485 using field equipment 411. For example, in this case, storage vessel 409-1 has field equipment 411-1 that contains a gas 485-1 within the storage vessel 409-1. As another example, in this case, storage vessel 409-X has field equipment 411-X that contains a gas 485-X within the storage vessel 409-X. For purposes herein, the field equipment 411 is considered part of the storage vessel 409. In this case, the field equipment 411-1 is considered part of the storage vessel 409-1, and the field equipment 411-X is considered part of the storage vessel 409-X.

In some cases, the field equipment 411 of a storage vessel 409 may fail, allowing some of the gas 485 within the storage vessel 409 to escape into the outside environment 494. As a result, the field equipment 411 shows signs of a failure 413 (e.g., failure 413-1 for the field equipment 411-1 of storage vessel 409-1, failure 413-X for the field equipment 411-X of storage vessel 409-X) leading to a leak point 412 (e.g., leak point 412-1 in the field equipment 411-1 of the storage vessel 409-1, leak point 412-X in the field equipment 411-X of the storage vessel 409-X) from which the leak in the outside environment 494 emanates. Each leak point 412 is sometimes called a source of the leak 401 (e.g., leak 401-1 from storage vessel 409-1, leak 401-X from storage vessel 409-X) or plume of the gas 485.

As discussed below, the example mobile vehicle 440 is configured to collect gas samples from the outside environment 494, filter the samples, measure the processed samples 472, and determine whether a gas 485 is leaking from a storage vessel 409 into the outside environment 494. Examples of a gas 485 may include, but are not limited to, dihydrogen, carbon dioxide, methane, helium, noble gases, water, water vapor, carbon monoxide, nitric oxide, nitrogen dioxide, sulfur dioxide, ozone, hydrogen sulfide, ammonia, and radiation. As defined herein, the outside environment 494 is any environment that is outside of a storage vessel 409. An outside environment may be outdoors or indoors (e.g., in a plant or manufacturing facility). The outside environment 494 may be in open air above ground (e.g., ground 108, ground 208, ground 308) or, in some cases, below ground in the subterranean formation (e.g., subterranean formation 110, subterranean formation 210, subterranean formation 310).

A user 451 may be any person that interacts, directly or indirectly, with the example mobile vehicle 440 and/or any other component of the system 400. Examples of a user 451 may include, but are not limited to, a business owner, an engineer, a company representative, a geologist, a consultant, an environmentalist, a contractor, and a manufacturer's representative. A user 451 may use one or more user systems 455, which may include a display (e.g., a GUI). A user system 455 of a user 451 may interact with (e.g., send data to, obtain data from) a controller 404 via an application interface and using the communication links 405. The user 451 may also interact directly with a controller 404 through a user interface (e.g., keyboard, mouse, touchscreen).

The network manager 480 is a device or component that controls all or a portion (e.g., a communication network, a controller 404) of the system 400. The network manager 480 may be substantially similar to some or all of a controller 404, as described below. For example, the network manager 480 may include a controller that has one or more components and/or similar functionality to some or all of a controller 404. Alternatively, the network manager 480 may include one or more of a number of features in addition to, or altered from, the features of a controller 404. As described herein, control and/or communication with the network manager 480 may include communicating with one or more other components of the same system 400 and/or another system. In such a case, the network manager 480 may facilitate such control and/or communication. The network manager 480 may be called by other names, including but not limited to a master controller, a network controller, and an enterprise manager. The network manager 480 may be considered a type of computer device, as discussed below with respect to FIG. 6.

Each sensor device 360 includes one or more sensors that measure one or more parameters (e.g., pressure, flow rate, temperature, humidity, voltage, current, location, distance, wind speed, wind direction, a position of a valve, a fluid level, barometric pressure, time, etc.). Examples of a sensor of a sensor device 360 may include, but are not limited to, a temperature sensor, a flow sensor, a pressure sensor, a gas spectrometer, a voltmeter, an ammeter, an anemometer, a hygrometer, a hydrometer, a spectrograph, a gas chromatograph, and a camera. A sensor device 360 may be a stand-alone device or integrated with another component (e.g., a motor, a valve, a circuit breaker) of the system 400.

A parameter measured by a sensor device 360 may be associated with something that is tangentially associated with the operation of the example mobile vehicle 440. In this way, a measurement made by a sensor device 360 may be used in conjunction with measurements made by the sensor devices 460 of the mobile vehicle 440 in the functions performed by a controller 404 of the analytic system 450. When a sensor device 360 includes its own controller 304 (or portions thereof), then the sensor device 360 may be considered a type of computer device, as discussed below with respect to FIG. 6.

Interaction between the sensor devices 460, the sensor devices 360, the controllers 304, the controllers 404 of the analytic system 450, the various components (e.g., the mobility features 495, the processing apparatus 444) within the example mobile vehicle 440, the users 451 (including any associated user systems 455), the network manager 480, and other components (e.g., valves, equipment associated with the storage vessels 409) of the system 400 may be conducted using communication links 405 and/or power transfer links 487. Each communication link 405 may include wired (e.g., Class 1 electrical cables, Class 2 electrical cables, electrical connectors, Power Line Carrier, RS485) and/or wireless (e.g., Wi-Fi, Zigbee, visible light communication, cellular networking, Bluetooth, Bluetooth Low Energy (BLE), ultrawide band (UWB), WirelessHART, ISA100) technology. A communication link 405 may transmit signals (e.g., communication signals, control signals, data) between the sensor devices 460, the sensor devices 360, the controllers 304, the controllers 404 of the analytic system 450, the various components (e.g., the mobility features 495, the processing apparatus 444) within the example mobile vehicle 440, the users 451 (including any associated user systems 455), the network manager 480, and the other components of the system 400.

Each power transfer link 487 may include one or more electrical conductors, which may be individual or part of one or more electrical cables. In some cases, as with inductive power, power may be transferred wirelessly using power transfer links 487. A power transfer link 487 may transmit power between the sensor devices 460, the sensor devices 360, the controllers 304, the controllers 404 of the analytic system 450, the various components (e.g., the mobility features 495, the processing apparatus 444) within the example mobile vehicle 440, the users 451 (including any associated user systems 455), the network manager 480, and the other components of the system 400. Each power transfer link 487 may be sized (e.g., 12 gauge, 18 gauge, 4 gauge) in a manner suitable for the amount (e.g., 480V, 24V, 120V) and type (e.g., alternating current, direct current) of power transferred therethrough.

Each of the controllers 304 of the system 400 is a device or component that controls a portion (e.g., a communication network, some of the equipment associated with one or more of the storage vessels 409) of the system 400. A controller 304 may be substantially similar to some or all of the controller 404, as described above. For example, a controller 304 may include a controller that has one or more components and/or similar functionality to some or all of a controller 404 (discussed below) of the analytic system 450 of the mobile vehicle 440. Alternatively, a controller 304 may include one or more of a number of features in addition to, or altered from, the features of a controller 404. As described herein, control and/or communication with a controller 304 may include communicating with one or more other components of the system 400 and/or another system. In such a case, a controller 304 may facilitate such control and/or communication. Each controller 304 may be considered a type of computer device, as discussed below with respect to FIG. 6.

As discussed above, the body 441 of the example mobile vehicle 440 is configured to protect one or more other components (e.g., a controller 404, some or all of the processing apparatus 444, some or all of the mobility features 495) of the mobile vehicle 440 while allowing the mobile vehicle 440 to move and operate. The body 441 can have any suitable configuration (e.g., shape, size) and/or be made of one or more of any suitable materials (e.g., plastic, stainless steel, aluminum, rubber, composite, glass). The body 441 may include one or more openings (e.g., permanent, retractable) to accommodate one or more other components (e.g., a sample collector 445, a mobility feature 495) of the mobile vehicle 440.

The sample collector 445 of the example mobile vehicle 440 is configured to collect samples that include air from the outside environment 494. When a storage vessel 409 has a failure of its field equipment 411 so that some of the gas 485 contained in the storage vessel 409 leaks, one or more of the samples collected by the sample collector 445 may include the gas 485. The sample collector 445 may collect samples from the outside environment 494 on a continuous basis or on a discrete basis (e.g., at regular intervals of time, upon the occurrence of an event, randomly). The sample collector 445 may collect samples using forced air, induced air, stabbing, scooping, and/or any other method suitable for collecting a sample.

The equipment included in the sample collector 445 may be suitable for collecting and securing each sample (also called an original sample 471 herein) taken from the outside environment 494. Examples of such equipment may include, but are not limited to, a chamber, a capsule, a column, a tube, and a flask. The sample collector 445 may be or include a single apparatus (with or without multiple portions) for collecting one or more samples or multiple apparatuses (or portions thereof) that operate in series and/or in parallel with each other.

When the sample collector 445 includes multiple (e.g., 2, 3, 5, 8, 11, 18, 25) apparatuses, the sample collector 445 may collect multiple samples simultaneously from different points in the outside environment 494 stemming from the body 441 of the mobile vehicle 440. In some cases, the sample collector 445 may include ancillary features (e.g., a temperature conditioning feature, a mixing feature, a drying feature, a separating feature) that may be used to preserve and/or process a sample. For example, the sample collector 445 may include a compressor, nitrogen, and/or a zero air addition to increase the pressure of a sample to greater than ambient air pressure (e.g., the pressure of the outside environment 494).

In some cases, an original sample 471 that is collected by the sample collector 445 includes a gas 485, even if there is no leak in an associated storage vessel 409. For example, if the gas 485-1 stored in the storage vessel 409-1 is dihydrogen, an original sample 471 of the air in the outside environment 494 includes some amount of dihydrogen, whether in pure form and/or as part of some other hydrogen-based compound (e.g., water), even if there is no leak in the storage vessel 409-1. As another example, if the gas 485-X stored in the storage vessel 409-X is carbon dioxide, an original sample 471 of the air in the outside environment 494 includes some amount of carbon dioxide and/or derivative therefor (e.g., methane), even if there is no leak in the storage vessel 409-X.

Each original sample 471 collected by a sample collector 445 may be correlated with information captured by one or more of the sensor devices 460 (discussed below) of the example mobile vehicle 440. For example, if a sensor device 460 is capable of measuring a GPS coordinate from where the sample collector is located, the GPS coordinates may be associated with an original sample 471 by a controller 404 (discussed below) of the analytic system 450 of the mobile vehicle 440. As another example, a time stamp generated by the timer 535 of a controller 404 may be associated with an original sample 471 by a controller 404 (discussed below) of the analytic system 450 of the mobile vehicle 440. Examples of at least a portion of an original sample 471 that is obtained by a sample collector 445 may include, but are not limited to, □¹³CH₄ (atmospheric methane), CH₄ (methane), C₂H₆ (ethane), CO₂ (carbon dioxide), CO (carbon monoxide), H₂O (water), NH₃ (ammonia), O₃ (ozone), SO₂ (sulfur dioxide), NOx (nitrogen oxides), NO (nitrogen oxide), NO₂ (nitrogen dioxide), N₂O (nitrous oxide), and H₂S (hydrogen sulfide).

The processing apparatus 444 of the example mobile vehicle 440 is configured to process an original sample 471 to generate a processed sample 472. For example, the processing apparatus 444 may be configured to remove (e.g., filter out) a naturally-occurring quantity of a gas 485 (e.g., in raw form, as a chemical compound that is derived from the gas 485) from an original sample 471 to generate a processed sample 472. For example, if the gas 485 is dihydrogen, then the processing apparatus 444 may be configured to remove (e.g., using a membrane and/or cryogenic process) the water and/or water vapor (and/or some other fluid) from the original sample 471 without removing H₂ to generate a processed sample 472. The processing apparatus 444 may additionally or alternatively be configured to remove (or at least reduce the amount of) dust, dirt, and/or other debris from an original sample 471.

The processing apparatus 444 may additionally or alternatively be configured to convert (e.g., using a fuel cell catalyst (platinum)) H₂ to water vapor within an original sample 471 to generate (or take a step toward generating) a processed sample 472. This other fluid may or may not include the gas 485, a derivation of the gas 485, and/or a component (e.g., carbon, hydrogen) of the gas 485. In addition, or in the alternative, the processing apparatus 444 may be configured to otherwise process (e.g., mix, heat, dry, cool, introduce an additive, pressurize, depressurize, dehumidify, hydrate, stimulate) some or all of one or more original samples 471 at a point in time and/or over a period of time.

To perform its one or more functions, a processing apparatus 444 may include any of a number of different equipment. For example, a processing apparatus 444 may include a membrane, a cryogenic apparatus, a heater, a cooler, a blower, a fan, a mixer, a centrifuge, a strainer, a separator, a funnel, an agitator, a bladder, a pump, a motor, a meter, a hydrogen fuel cell or similar catalyst, a sensor device (e.g., sensor device 460), a controller (e.g., controller 404), and a compressor. The processing apparatus 444 may be controlled by a user 451 (e.g., a human being), by a controller 404 of the analytic system 450, by its own controller (e.g., similar to a controller 404), some other entity, or any combination thereof.

The processing apparatus 444 may operate substantially continuously (as when the original samples 471 substantially continuously flow into the processing apparatus 444) or at intervals (as when the original samples 471 are introduced into the processing apparatus 444 intermittently). The processing apparatus 444 may be or include a single apparatus (with or without multiple portions) or multiple apparatus (or portions thereof) that operate in series and/or in parallel with each other. As an example, the processing apparatus 444 may include a temperature conditioning portion, a drying portion, and a separating portion that operate in series with each other. As another example, the processing apparatus 444 may include multiple separators that operate in parallel with each other, where each separator may separate one or more fluids (e.g., a gas 485) from an original sample 471 or variation thereof simultaneously. The processing apparatus 444 may control various aspects (e.g., temperature, pressure) of the original samples 471 (or partially processed portions thereof). In some cases, the processing apparatus 444 is designed to subject the original samples 471 (or partially processed portions thereof) to conditions (e.g., pressure, temperature) that are designed to optimize test results.

In some cases, some or all of the processing apparatus 444 may be operated, paused, and/or stopped so that the original samples 471 (or partially processed portions thereof) may be converted into processed samples 472 for evaluation by the analytic system 450. Testing of processed samples 472 by the analytic system 450 may be controlled by a user 451 (e.g., a human being) and/or a controller 404 of the analytic system 450. Testing of processed samples 472 by the analytic system 450 may be based on historical data and/or field data (e.g., measurements from sensor devices 460). Testing of processed samples 472 by the analytic system 450 may generate results that indicate whether there is an excessive amount of a gas 485, indicating that a storage vessel 409 has a leak. Testing of processed samples 472 by the analytic system 450 may be conducted using one or more algorithms 533, one or more protocols 532, and/or stored data 534 (all discussed below).

The mobility features 495 of the mobile vehicle 440 are devices and/or components that allow the mobile vehicle 440 to move. The mobile vehicle 440 can have one or more of any number and/or type of mobility features 495. Examples of such mobility features 495 can include, but are not limited to, wheels, propellers, caterpillar tracks, grippers, spikes, anchors, motors, axels, gears, a heat sink, an electrical conductor or electrical cable, a terminal block, a drive train, and a circuit board. In this way, the mobile vehicle 440 can move along the ground (e.g., ground 208), through the air, in liquid (e.g., in a lake, in a pond, in an ocean), up and down a wall (e.g., of a tank or similar form of storage vessel 409) or a hill, and/or around obstacles (e.g., boulders, buildings, equipment) in the outside environment 494. The mobile vehicle 440 can be a car, a truck, a crawler, a submersible vehicle, a drone, a hovercraft, and/or any other type of movable device.

In certain example embodiments, one or more optional repair features 443 of the mobile vehicle 440 are configured to perform or facilitate the performance of repairs and/or other actions designed to fix a leak point 412 and/or a failure 413 in a storage vessel 409 (including field equipment 411 thereof). Examples of a repair feature 443 may include, but are not limited to, a mechanical arm, a drill, a mechanical screwdriver, an epoxy applicator, a spray mechanism, an epoxy, a wrench, a hammer, a replacement valve, welding equipment, a gasket, a metal plate, and piping. One or more of the repair features 443, when present, may be controlled by a controller 404 of the mobile vehicle 440 using stored data 534, one or more protocols 532, one or more algorithms 533, measurements from one or more of the sensor devices 460, and/or input from a user 451 (including an associated user system 455). In addition, or in the alternative, one or more of the repair features 443, when present, may be controlled by a user 451. The repair features 443 allow for the reduction or elimination of a leak 401 in real time when the leak 401 is detected by the mobile vehicle 440.

The power supply 456 of the mobile vehicle 440 can include one or more components (e.g., a transformer, a diode bridge, an inverter, a converter) that receives power (for example, through an electrical cable) from a source (e.g., a battery, fuel, a power source external to the mobile vehicle 440) and generates power of a type (e.g., alternating current, direct current) and level (e.g., 12V, 24V, 120V) that can be used by the one or more controllers 404, the mobility features 495, and/or some or all of the other components of the mobile vehicle 440. In addition, or in the alternative, the power supply 456 can be or include a source of power in itself. For example, the power supply 456 can be or include a battery or some other source of independent power (e.g., solar panels). In some cases, a controller 404 can generate and send a signal to the power supply 456 to control the operation and/or output of the power supply 456.

The analytic system 450 of the mobile vehicle 440 may be configured to perform a composition analysis of the processed samples 472 derived from the original samples 471 obtained by the sample collectors 445 from the outside environment 494. In addition, the analytic system 450 of the mobile vehicle 440 may be configured to determine, based on performing a composition analysis of the processed samples 472, whether one or more of the storage vessels 409 has a leak. As a result, the mobile vehicle 440 (and more specifically the analytic system 450) may be used to evaluate multiple storage vessels 409 using composition analysis of the processed samples 472. As a result, example embodiments may be used, for example, to determine the location and/or severity of a leak in a particular storage vessel 409.

As discussed above, the analytic system 450 may include multiple components. For example, in this case, the analytic system 450 includes one or more testing apparatuses 470 and one or more controllers 404. Each testing apparatus 470 of the analytic system 450 may include one or more testing vessels 473 and one or more of the sensor devices 460. Each testing apparatus 470 may be configured to test one or more of the processed samples 472. A single testing apparatus 470 may perform multiple tests (e.g., on a single processed sample 472, on multiple processed samples 472) simultaneously.

When the analytic system 450 has multiple testing apparatuses 470, one testing apparatus 470 may operate in conjunction with, or independently of, one or more of the other testing apparatuses 470. Further, when the analytic system 450 has multiple testing apparatuses 470, one testing apparatus 470 may be configured (e.g., in terms of equipment, in terms of operating capability) the same as, or differently than, one or more of the other testing apparatuses 470. The operation of a testing apparatus 470 may be controlled by a user 451 (including an associated user system 455) and/or a controller 404 of the analytic system 450.

A testing vessel 473 of a testing apparatus 470 may be configured to retain a processed sample 472 for a period of time so that the composition of the processed sample 472 may be tested and analyzed. A testing vessel 473 of a testing apparatus 470 may be a man-made storage tank or other type of vessel (e.g., a bottle, a column, a test tube, a chamber). A testing vessel 473 of a testing apparatus 470 may be configured to hold a solid, a liquid, and/or a gas. A testing vessel 473 of a testing apparatus 470 may be configured to accommodate any of a number of parameters (e.g., pressure, temperature, acid or base content) used to receive, store, condition, test, and/or analyze the processed sample 472. In some cases, a testing vessel 473 (or other portion of the testing apparatus 470) may include shock absorbers and/or similar equipment to stabilize the testing vessel 473 (or at least the processed sample 472 therein) during times of vibration and/or other types of events that may disrupt and/or skew the results of testing on a processed sample 472.

A testing apparatus 470 may include or interact with one or more sensor devices 460 (discussed below) to perform one or more of its functions. Testing performed by a testing apparatus 470 may use or include historical data and/or field data (e.g., measurements from sensor devices 460). Testing may yield a quantification of one or more gases 485. When an amount of a gas 485 exceeds a threshold or acceptable value, as determined by a controller 404 of the testing apparatus 470, a controller 404 may determine whether the gas 485 originates from one or more of the storage vessels 409.

A controller 404 of the analytic system 450 may be configured to evaluate, using measurements obtained from the sensor devices 460, including measurements of one or more parameters associated with one or more processed samples 472, whether one or more of the storage vessels 409 has a leak. In some cases, a controller 404 of the analytic system 450 may also be configured to identify the source of the leak in the storage vessel 409, determine the extent of the leak, identify steps that may be taken to reduce or eliminate the leak, and/or control one or more actions taken by the mobile vehicle 440 to reduce or eliminate the leak.

The analytic system 450 may be configured to process and/or test a processed sample 472. The analytic system 450 may include one or more of any of a number of different equipment, including but not limited to a sifter, a compressor, a membrane, a chiller, a fan, a pump, a motor, a controller (e.g., controller 404), and a sensor device (e.g., sensor device 460). In some cases, the analytic system 450, or portions thereof, may operate using a controller 404. In addition, or in the alternative, one or more users 451 may perform one or more of the various functions required to operate some or all of the analytic system 450. The analytic system 450 may be used in conjunction with one or more sensor devices 460. The analytic system 450 may be or include a vessel (e.g., a bottle, a column, a test tube) inside of which one or more of the processed samples 472 are disposed for testing.

Each sensor device 460 of the mobile vehicle 440 includes one or more sensors that measure one or more parameters (e.g., pressure, flow rate, temperature, humidity, voltage, current, location, distance, wind speed, wind direction, chemical composition of a processed sample 472, barometric pressure, time, aerosol profiles, and aerosol size spectra (e.g., 0.1-32 μm, 32 channels, 0.16 Hz), etc.). Examples of a sensor of a sensor device 460 may include, but are not limited to, a cavity ring-down spectrometer (CRDS), a temperature sensor, a flow sensor, a pressure sensor, a gas chromatograph, a gas spectrometer, a mass spectrometer, Raman spectrometer, a voltmeter, an ammeter, an anemometer (e.g., 3D sonic), an inclinometer, a fluorescence analyzer, an absorption analyzer, a hygrometer, a hydrometer, a spectrograph, and a camera. A sensor device 460 may be a stand-alone device or integrated with another component (e.g., the processing apparatus 444) of the mobile vehicle 440.

A parameter measured by a sensor device 460 may be associated with a processed sample 472. In some cases, in addition, a parameter measured by a sensor device 460 may be associated with the mobile vehicle 440 (e.g., current speed and/or direction of movement, amount of vibration), the outside environment 494 (e.g., wind speed, wind direction, 3D winds, temperature, humidity, atmospheric pressure), a storage vessel 409 (e.g., surface temperature, distance relative to the mobile vehicle 440), and/or some other component of the system 400. A sensor device 460 may be configured to detect and/or measure one or more fluids (e.g., atmospheric methane, methane, ethane, carbon dioxide, carbon monoxide, water vapor, liquid water, ammonia, ozone, sulfur dioxide, nitrogen oxides, nitrogen oxide, nitrogen dioxide, nitrous oxide, hydrogen sulfide) at sub ppb (below parts per billion) levels in an original sample 471 and/or a processed sample 472. A sensor device 460 may be configured to detect and/or measure one or more fluids (e.g., carbon dioxide, water vapor, liquid water) in ppm (parts per million).

In some cases, a number of sensor devices 460, each measuring a different parameter, may be used in combination to determine and confirm whether a controller 404 of the analytic system 450 and/or a controller 304 should take a particular action (e.g., operate a valve, operate or adjust the operation of a mobility feature 495, operate or adjust the operation of the processing apparatus 444, operate or adjust the operation of a sample collector 445). When a sensor device 460 includes its own controller 404 (or portions thereof), then the sensor device 460 may be considered a type of computer device, as discussed below with respect to FIG. 6.

In certain example embodiments, a sensor device 460 and/or other portions (e.g., a mobility feature 495) of the mobile vehicle 440 is configured to compensate or correct for any of a number of external factors (e.g., dust, vibrations) that may influence the measurement of a parameter associated with a processed sample 472 and/or the outside environment 494 made by the sensor device 460. In this way, a measurement made by a sensor device 460 is accurate despite the presence of dust, vibrations, and/or other factors that would otherwise lead to a different value.

As discussed above, the analytic system 450 may include one or more controllers 404. A controller 404 of the analytic system 450 communicates with and in some cases controls one or more of the other components (e.g., a sensor device 460, a sample collector 445, the processing apparatus 444, a mobility feature 495, another controller 404) of the example mobile vehicle 440 and/or one or more other components (e.g., a sensor device 360, a controller 304, one or more valves) of a remainder of the system 400. A controller 404 performs any of a number of functions that include, but are not limited to, obtaining and sending data, evaluating data, following protocols, running algorithms, and sending commands.

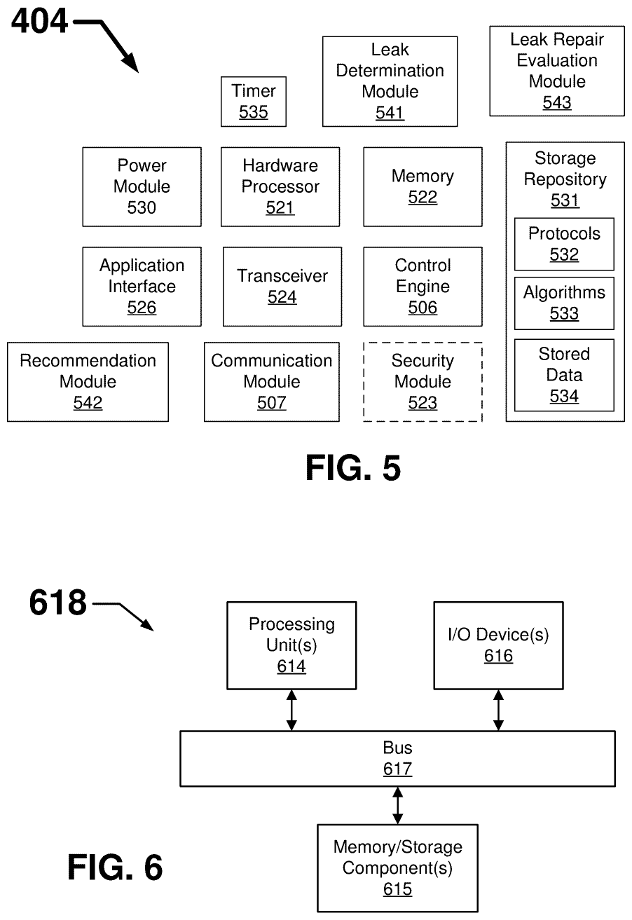

A controller 404 may include one or more of a number of components. For example, as shown in FIG. 5, such components of a controller 404 may include, but are not limited to, a control engine 506, a leak determination module 541, a recommendation module 542, a leak repair evaluation module 543, a communication module 507, a timer 535, a power module 530, a storage repository 531, a hardware processor 521, a memory 522, a transceiver 524, an application interface 526, and, optionally, a security module 523. A controller 404 (or components thereof) may be located at or near the various components of the mobile vehicle 440. In addition, or in the alternative, the controller 404 (or components thereof) may be located remotely from (e.g., in the cloud, at an office building) the various components of the mobile vehicle 440.

When there are multiple controllers 404 (e.g., one controller 404 for a sample collector 445, another controller 404 for a mobility feature 495, yet another controller 404 for the processing apparatus 444, still another controller 404 for the analytic system 450), each controller 404 may operate independently of each other. Alternatively, two or more of the multiple controllers 404 may work cooperatively with each other. As yet another alternative, one of the controllers 404 may control some or all of one or more other controllers 404 in the system 400 or portion thereof. Each controller 404 may be considered a type of computer device, as discussed below with respect to FIG. 6.

The storage repository 531 of a controller 404 may be a persistent storage device (or set of devices) that stores software and data used to assist a controller 404 in communicating with one or more other components of a system, such as the users 451 (including associated user systems 455), one or more of the mobility features 495, the processing apparatus 444, one or more of the sample collectors 445 the controllers 304, the sensor devices 360, other controllers 404 of the analytic system 450, the network manager 480, the sensor devices 460, etc. of the system 400 of FIG. 4 above. In one or more example embodiments, the storage repository 531 stores one or more protocols 532, one or more algorithms 533, and stored data 534.

The protocols 532 of the storage repository 531 may be any procedures (e.g., a series of method steps) and/or other similar operational processes that the control engine 506 of a controller 404 follows based on certain conditions at a point in time. The protocols 532 may include any of a number of communication protocols that are used to send and/or obtain data between a controller 404 and other components of a system (e.g., the system 400). Such protocols 532 used for communication may be time-synchronized protocols. Examples of such time-synchronized protocols may include, but are not limited to, a highway addressable remote transducer (HART) protocol, a WirelessHART protocol, and an International Society of Automation (ISA) 100 protocol. In this way, one or more of the protocols 532 may provide a layer of security to the data transferred within a system (e.g., the system 400). Other protocols 532 used for communication may be associated with the use of Wi-Fi, Zigbee, visible light communication (VLC), cellular networking, BLE, UWB, and Bluetooth.

The algorithms 533 may be any formulas, mathematical models, forecasts, simulations, and/or other similar tools that the control engine 506 of a controller 404 uses to reach a computational or logical conclusion. For example, one or more algorithms 533 may be used, in conjunction with one or more protocols 532, to assist a controller 404 to determine when to start, adjust, and/or stop the operation of a sample collector 445, a mobility feature 495, the processing apparatus 444, a sensor device 460, another controller 404 of the analytic system 450, and/or another component of the system 400. As another example, one or more algorithms 533 may be used, in conjunction with one or more protocols 532, to assist a controller 404 to determine when to have a sensor device 460 measure a parameter and subsequently assist the controller 404 in performing a calculation or make a determination (e.g., detect a leak of a gas 485 from a storage vessel 409) using the measurement.

As yet another example, one or more algorithms 533 may be used, in conjunction with one or more protocols 532, to assist a controller 404 to determine where additional samples should be collected by one or more of the sample collectors 445. As still another example, one or more algorithms 533 may be used, in conjunction with one or more protocols 532, to assist a controller 404 to identify a storage vessel 409 that is leaking a gas 485. As yet another example, one or more algorithms 533 may be used, in conjunction with one or more protocols 532, to assist a controller 404 in determining how a leak in a storage vessel 409 may be slowed or stopped. As still another example, one or more algorithms 533 may be used, in conjunction with one or more protocols 532, to assist a controller 404 to take actions to slow or stop a leak of a gas 485 in a storage vessel 409.

Stored data 534 may be any data associated with a gas 485 and/or a storage vessel 409, the components (e.g., the body 441, the sample collectors 445, the mobility features 495, the processing apparatus 444, the sensor devices 460) of the mobile vehicle 440, the other components (e.g., the user systems 455, the power transfer links 487, the communication links 405, the sensor devices 360, the controllers 304), including associated equipment (e.g., motors, pumps, compressors), of the rest of the system 400, measurements made by the sensor devices 460 and the sensor devices 360, prior repairs made to the storage vessels 409 (including dates of the repairs, location of the repairs with respect to the storage vessels 409, actions taken in the repairs), threshold values, tables, results of previously run or calculated algorithms 533, updates to protocols 532, user preferences, and/or any other suitable data. Such data may be any type of data, including but not limited to historical data, present data, and future data (e.g., forecasts). The stored data 534 may be associated with some measurement of time derived, for example, from the timer 535.

Examples of a storage repository 531 may include, but are not limited to, a database (or a number of databases), a file system, cloud-based storage, a hard drive, flash memory, some other form of solid-state data storage, or any suitable combination thereof. The storage repository 531 may be located on multiple physical machines, each storing all or a portion of the communication protocols 532, the algorithms 533, and/or the stored data 534 according to some example embodiments. Each storage unit or device may be physically located in the same or in a different geographic location.

The storage repository 531 may be operatively connected to the control engine 506. In one or more example embodiments, the control engine 506 of a controller 404 includes functionality to communicate with the users 451 (including associated user systems 455), the sample collectors 445, the mobility features 495, the processing apparatus 444, the other controllers 404, the sensor devices 460, the sensor devices 360, the controllers 304, the network manager 480, and/or the other components in the system 400, including the example mobile vehicle 440. More specifically, the control engine 506 sends information to and/or obtains information from the storage repository 531 in order to communicate with the users 451 (including associated user systems 455), the sample collectors 445, the mobility features 495, the processing apparatus 444, the other controllers 404, the sensor devices 460, the sensor devices 360, the controllers 304, the network manager 480, and/or the other components of the system 400, including the example mobile vehicle 440. As discussed below, the storage repository 531 may also be operatively connected to the communication module 507 in certain example embodiments.

In certain example embodiments, the control engine 506 of a controller 404 controls the operation of one or more components (e.g., the communication module 507, the timer 535, the transceiver 524) of the controller 404. For example, the control engine 506 may activate the communication module 507 when the communication module 507 is in “sleep” mode and when the communication module 507 is needed to send data obtained from another component (e.g., a sensor device 460) in the system 400, including the example mobile vehicle 440. In addition, the control engine 506 of a controller 404 may control the operation of one or more other components (e.g., the sample collectors 445, the mobility features 495, the processing apparatus 444, the other controllers 404), or portions thereof, of the system 400, including the example mobile vehicle 440.

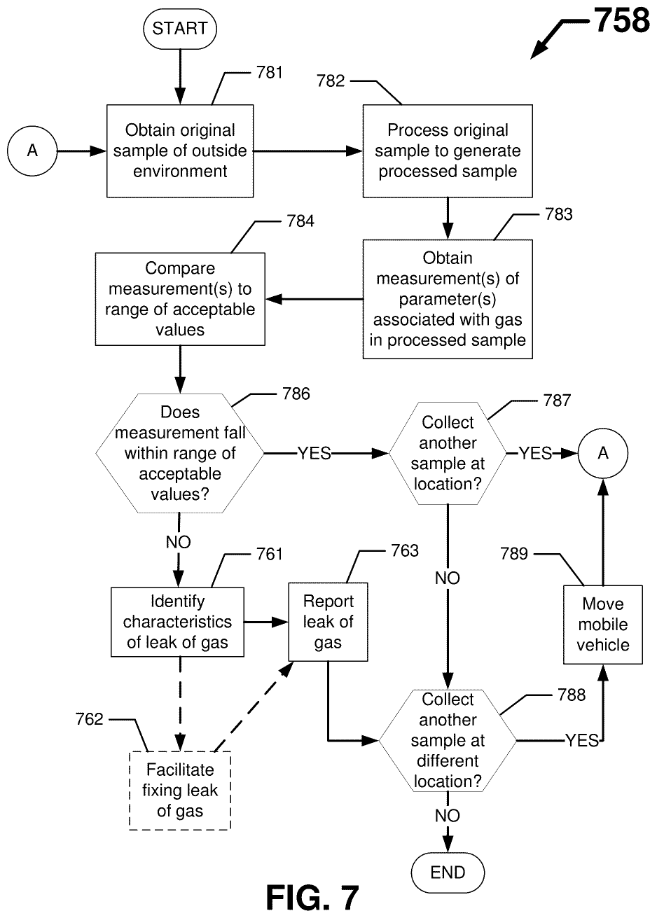

The control engine 506 of a controller 404 may communicate with one or more other components of the system 400 and/or an external system. For example, the control engine 506 may use one or more protocols 532 to facilitate communication with the sensor devices 360 to obtain data (e.g., measurements of various parameters, such as gas content, temperature, pressure, wind speed, wind direction, and flow rate), whether in real time or on a periodic basis and/or to instruct a sensor device 460 to take a measurement. As yet another example, the control engine 506 may use one or more algorithms 533 and/or protocols 532 to generate a new or updated algorithm 533 and/or a new or updated protocol 532 based on actual results compared to expected results. A number of other capabilities of the control engine 506 (as well as the controller 404 as a whole and/or other portions of the controller 404) are discussed below with respect to FIG. 7.

The control engine 506 may generate and process data associated with control, communication, and/or other signals sent to and obtained from the users 451 (including associated user systems 455), the sensor devices 460, the sensor devices 360, the controllers 304, the other controllers 404, the sample collectors 445, the mobility features 495, the processing apparatus 444, the network manager 480, and the other components of the system 400, including the example mobile vehicle 440. In certain embodiments, the control engine 506 of the controller 404 may communicate with one or more components of a system external to the system 400. For example, the control engine 506 may interact with an inventory management system by ordering replacements for components or pieces of equipment (e.g., a sensor device 460, a valve, a storage vessel 409) within the system 400 that has failed or is failing. As another example, the control engine 506 may interact with a contractor or workforce scheduling system by arranging for the labor needed to replace a component or piece of equipment in the system 400. In this way and in other ways, the controller 404 is capable of performing a number of functions beyond what could reasonably be considered a routine task.

In certain example embodiments, the control engine 506 may include an interface that enables the control engine 506 to communicate with the sensor devices 460, the sensor devices 360, the controllers 304, the other controllers 404, the sample collectors 445, the mobility features 495, the processing apparatus 444, the user systems 455, the network manager 480, and/or other components of the system 400, including the example mobile vehicle 440. For example, if a user system 455 operates under IEC Standard 62386, then the user system 455 may have a serial communication interface that will transfer data to the controller 404. Such an interface may operate in conjunction with, or independently of, the protocols 532 used to communicate between the controller 404 and the users 451 (including corresponding user systems 455), the sensor devices 460, the sensor devices 360, the controllers 304, the other controllers 404, the sample collectors 445, the mobility features 495, the processing apparatus 444, the network manager 480, and the other components of the system 400, including the example mobile vehicle 440.

The control engine 506 (or other components of the controller 404) may also include one or more hardware components and/or software elements to perform its functions. Such components may include, but are not limited to, a universal asynchronous receiver/transmitter (UART), a serial peripheral interface (SPI), a direct-attached capacity (DAC) storage device, an analog-to-digital converter, an inter-integrated circuit (I2C), and a pulse width modulator (PWM).

The leak determination module 541 of the controller 404 may be configured to determine a leak of a gas 485 from a storage vessel 409 into the outside environment 494. For example, the leak determination module 541 may use measurements of parameters taken by one or more of the sensor devices 460, where the parameters are associated with a gas 485 and/or a storage vessel 409. Using stored data 534 (e.g., prior measurements, threshold values, baseline values), one or more protocols 532 and/or one or more algorithms 533, the leak determination module 541 may determine the existence of a leak of a gas 485 in a storage vessel 409, the extent of the leak of the gas 485 in the storage vessel 409, and/or the location of the leak of the gas 485 in the storage vessel 409.

Implementation of the functions of the leak determination module 541 may be performed in one or more of a number of ways. For example, the leak determination module 541 may determine a difference in the value of at least one parameter between a baseline (e.g., ambient air with no leaks in any storage vessels 409) and results of testing filtered samples or portions thereof (e.g., a gas 485) proximate to at least one of the storage vessels 409, where the difference exceeds a threshold parameter value (e.g., part of the stored data 534) for the at least one parameter. In some cases, the leak determination module 541 may establish and/or maintain baseline values or ranges of baseline values for one or more parameters associated with a storage vessel 409 and/or the outside environment 494.

In certain example embodiments, the leak determination module 541 is further configured to use one or more algorithms 533, one or more protocols 532, and/or stored data 534 to perform one or more of its functions. For example, the leak determination module 541 may use a Gaussian plume model inversion to derive emissions based on filtered absolute winds (processed) and plume concentration anomalies. In such a case, a Gaussian Data Model (GDM) may be applied to the concentration anomaly, C′, relative to ambient, C. C′ may be calculated by subtracting the background as determined by a least-squares linear regression analysis to fit a first-order polynomial to concentrations outside the plume on both sides. This removes the effect of gradients but does not remove non-linear gradients. The following equation is an example of such an algorithm 533:

$${C}^{′} ( x , y , z ) = {E}_{A} \frac{exp ( \frac{- {y}^{2}}{2 {σ}_{y}^{2}} )}{( 2 π u {σ}_{z} {σ}_{y} )} ( exp ( \frac{- {( z - h )}^{2}}{2 {σ}_{z}^{2}} ) + exp ( \frac{- {( z + h )}^{2}}{2 {σ}_{z}^{2}} ) + exp ( \frac{- {( z - 2 ( B L - h ) )}^{2}}{2 {σ}_{z}^{2}} ) + exp ( \frac{- {( z + 2 ( B L - h ) )}^{2}}{2 {σ}_{z}^{2}} ) )$$

Then, C′ is projected onto a wind-orthogonal plane and least-squares fitted with one or more Gaussian functions (e.g., the Gaussian Plume Model—GPM). The GPM relates atmospheric emissions, E_{A}, to C′, wind speed, u, and the atmospheric turbulence parameters, σ_{y} and σ_{z}, defined in a cartesian coordinate system where x is the downwind, direction, θ, y is the transverse direction, and z is the vertical coordinate. Also, h is the release height (after buoyant rise). The second exponential term represents reflection off the ground (e.g., ground 308) and assumes a non-sticky molecule. CO₂ and CH₄ are non-sticky molecules. Slightly sticky molecules include CO and ammonia (an indicator of husbandry CH₄ and CO₂ plumes, which can be a confounder where husbandry shares space with oil production). The third term represents reflection off the marine boundary layer at height BL, and the fourth term represents re-reflection off the ground.

The parameterization of σ_{y} and σ_{z} may be determined by the following equation, with a, b, and c depending on the stability class.

$${σ}_{y} = {a ( 1 + 1 {0}^{- 4} x )}^{- 1 / 2} ; {σ}_{z} = {b ( 1 + c x )}^{n}$$

As an example, as shown in the table below, stability class may be based on solar insolation, I, and u. Specifically, the following table shows turbulence parameters for atmospheric turbulence parameterizations for different stability classes. The turbulence parameterizations captured in the above equation are discrete, which introduces uncertainty in σ_{y} and σ_{z}, such as at stability class transitions.

| Class | | a | b | c | n | Class Description |
|---|---|---|---|---|---|---|
| A | open | 0.22 | 0.20 | 0 | — | Extremely unstable |
| B | open | 0.16 | 0.12 | 0 | — | Moderately unstable |
| C | open | 0.11 | 0.08 | 2 × 10⁻⁴ | −0.5 | Slightly unstable |
| D | open | 0.08 | 0.06 | 1.5 × 10⁻³ | −0.5 | Neutral |
| E | open | 0.06 | 0.03 | 3 × 10⁻⁴ | −1 | Slightly stable |
| F | open | 0.04 | 0.016 | 3 × 10⁻⁴ | −1 | Moderately stable |

To remove discretization distortions, a 2^{nd}-order polynomial may be fit to the turbulence parameters a, b, and c, for each insolation class with respect to u. In such a case, the polynomial fit may be evaluated for u (wind speed) to determine a, b, and c for all insolation classes. Given the coarse nature of the Pasquill stability classes, rather than trying to optimize, the center u for each class may be used in the parameterization.

The GDM is for a passive dispersant and assumes negligible along-wind diffusion, that u, σ_{y}, and σ_{z} are vertically and horizontally uniform along its trajectory, that u fluctuations are zero, and that u remains parallel to the x-axis (no veering). These assumptions imply an idealized, flat terrain of homogeneous roughness. Violations of these conditions may occur in terrestrial settings and may be addressed by model modification. Although the GDM provides upwind distance to the source, it does not account for wind veering, which can be assessed by triangulation, by comparison with remote sensing data, and/or by comparison with the location of infrastructure with the potential to leak. Thus, source location may be improved through the analysis of repetition, transect data, and/or from using other gas and infrastructure information.

Emissions and source location uncertainty may be assessed by Monte Carlo (MC) simulations (types of algorithms 533) based on measured parameter variability. For example, normalized distributions of parameters for the plume inversion model (wind speed, u, wind direction, θ, ambient background gas concentration, C) may be created with a standard deviation and mean based on in situ field measurements during the plume transect. For each model run, plume emissions and source distance may be optimized for the maximum correlation coefficient, R², between the measured and modeled concentration anomaly, C. The parameter distributions may then be subsampled randomly as initial conditions for the plume inversion model (a type of algorithm 533). A large number (e.g., 10,000) of MC simulations may be run to calculate distributions of emissions and source distances. Uncertainty in emissions and source distance may then be defined based on where each distribution decreases to 1/e of the maximum.

When some or all of the mobility features 495, some other component of the mobile vehicle 440, and/or nearby equipment in the outside environment 494 operate using internal combustion, CO₂ anomalies can arise in the original samples that are collected. Data obtained by the mobile vehicle 440 may provide multiple approaches to identify false positive anomalies unrelated to leakage, the most prominent being CO. Factors that may contribute to self-contamination may include, but are not limited to, rapid deceleration of the mobile vehicle 440, slow travel of the mobile vehicle 440 under a strong tailwind, and circling back downwind (which allows the mobile vehicle 440 to traverse its own exhaust plume downwind). The first two of these listed factors may be identified from wind data, motion data of the mobile vehicle 440, and CO₂ data, all measured by one or more sensor devices 460 of the mobile vehicle 440.

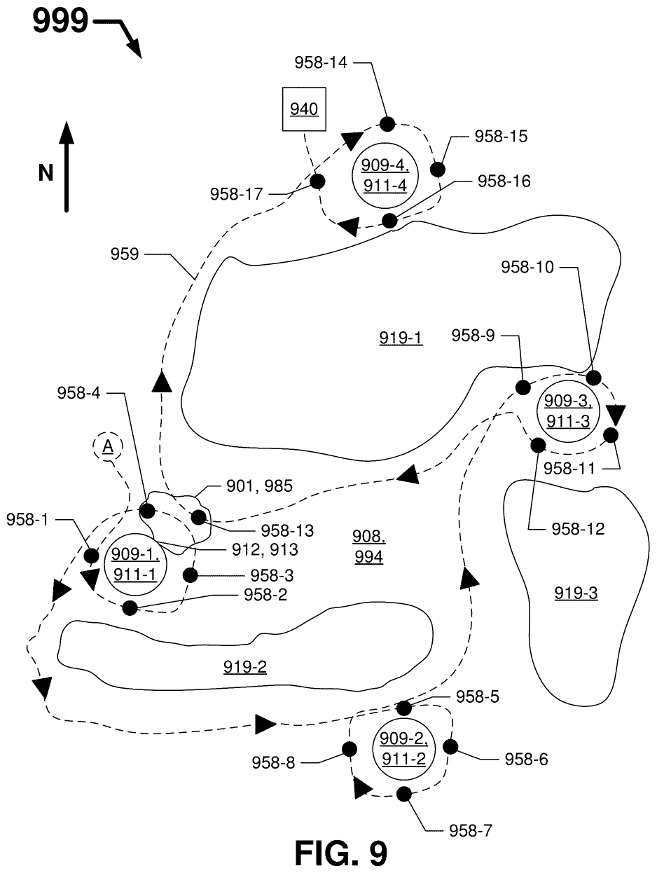

A downwind exhaust plume traverse, however, may be less obvious to detect and make corrections, particularly if there is wind veering. Strong orographic wind veering can occur in an outside environment 494 that includes hills (e.g., the hills 919 in FIG. 9 below) and/or other similar geographic features. Unexpected wind veering can drive an exhaust plume across the path of the mobile vehicle 440, elevating CO₂ significantly. In such cases, the exhaust signature of the mobile vehicle 440 may be confirmed by the measured amounts of CO in the original samples 471 and/or the processed samples 472. In some cases, a CO₂ plume may have no evident CH₄ anomaly but may be accompanied by water vapor and elevated aerosol concentrations, e.g., PM_{2.5}. In such cases, the CO′ plume (consistent with the slower PM_{2.5} data) may indicate that exhaust contamination spans a broader area than the CO₂′ data suggests. The water vapor may show a consistent pattern with CO, but the CO appears to show some latency, which is a problem for sticky molecules from adsorption/desorption on sample path walls.

Accurate assessment of the location and strength of the emission source (e.g., the leak point 412, the failure 413) by the leak determination module 541 may depend on correctly assessing the number of dominant sources. For example, an asymmetry in a CO₂ plume may suggest multiple major proximate sources, as does the CH4′ profiles' asymmetry. Comparison of the upwind and downwind winds show divergence upwind of a storage vessel 409 (or field equipment 411 thereof) and a wind speed gradient in the downwind transect, implying three-dimensionality in the winds and wind acceleration. In such cases, the larger CO₂′ and CH₄′ plumes may not be co-located, though there are small co-signatures in a single transect, and the structure may be difficult to model using one or more of the algorithms 533.

In some cases, the transect may be repeated multiple times under different winds. For example, there may be a small CO₂′ plume (manifesting as a “shoulder” or inflection) at a transect distance where the dominant CH₄′ plume is located. Inversion modeling may be used to derive emissions from the plumes. In cases where steam injection is used to mobilize reservoir oil, leaks from steam transport infrastructure may also leak CH₄ and CO₂. In such cases, the leak determination module 541 may use H₂O′ to fingerprint CH₄′ plumes as steam-related, resulting in a correspondence between the H₂O′ and CH₄′ plumes. A Gaussian plume inversion used by the leak determination module 541 may be sensitive to wind speed, wind direction, and the measured concentration profile, all of which vary due to natural processes. Uncertainty in CO₂ may be determined by Monte Carlo simulations used by the leak determination module 541.

Minimum detection of a plume requires measurements above a minimum noise level. The leak determination module 541 may use one or more algorithms 533 to calculate and analyze the probability distribution function (PDF) for anomaly concentration data to identify the noise level. For example, emissions from sources (plumes) may have a distinct PDF from the PDF of the noise, which describes the low concentration limit. Noise may arise from turbulence and transport irregularities, natural variability, analyzer noise, and/or other factors. In some cases, the noise level is the concentration where the PDF transitions between noise and plume.

Plume detection by the leak determination module 541 requires measurements of original samples 471 and/or processed samples 472 above the noise level, estimated at the single data point level from histograms (e.g., ~0.7 ppm). This noise estimate may be conservative, as any feature would likely cover multiple pixels except in the extreme near field, which improves SNR. Gaussian plume simulations may be conducted by the leak determination module 541 to investigate factors affecting detection. These simulations may assume CO₂ emissions at some distance (e.g., 1 m) above the ground (e.g., ground 208) with measurements made at a height (e.g., 3 m) based on the location of the sample collectors 445.

In some cases, for a set distance D, increasing wind speed u rapidly decreases peak concentration C_{Peak} due to dilution from greater airflow and greater dispersion. In some cases, for a set wind speed u, peak concentration C_{Peak} may increase and then decrease with D. In such cases, this pattern may arise from the vertical evolution of the plume, which may depend strongly on atmospheric stability simulated based on solar insolation and winds. Given that higher insolation corresponds to faster vertical mixing, the increase and decrease in C_{Peak} with D may be more rapid for the higher insolation case.

In some cases, detection scales with emissions, and so smaller emissions may require the collection of original samples closer to the leak point 412 and/or observations at lower wind speed (and/or lower insolation). Lower wind speeds may allow the detection of weaker plumes. However, in some cases there is a practical low wind limit (e.g., around 1.0-1.5 meters per second, corresponding to ~7.5 kg per day (or 45 cm³ per second) at a transect distance of 10-15 m from the leak point 412) where winds become unsteady and variable.

Regardless of the gas 485 at issue, there may be some amount of the gas 485 naturally occurring in the outside environment 494. Example embodiments are configured to distinguish a leak 401 of a gas 485 from these naturally-occurring amounts. For example, if the gas 485 is carbon dioxide, the leak determination module 541 may be configured to distinguish a leak 401 of CO₂ from other CO₂ sources, including but not limited to CO₂ (not sequestered) associated with the subterranean formation (e.g., subterranean formation 110), vehicles using combustion engines, on-site combustion, nearby CO₂ sources (e.g., manufacturing facilities), and more remote CO₂ sources.

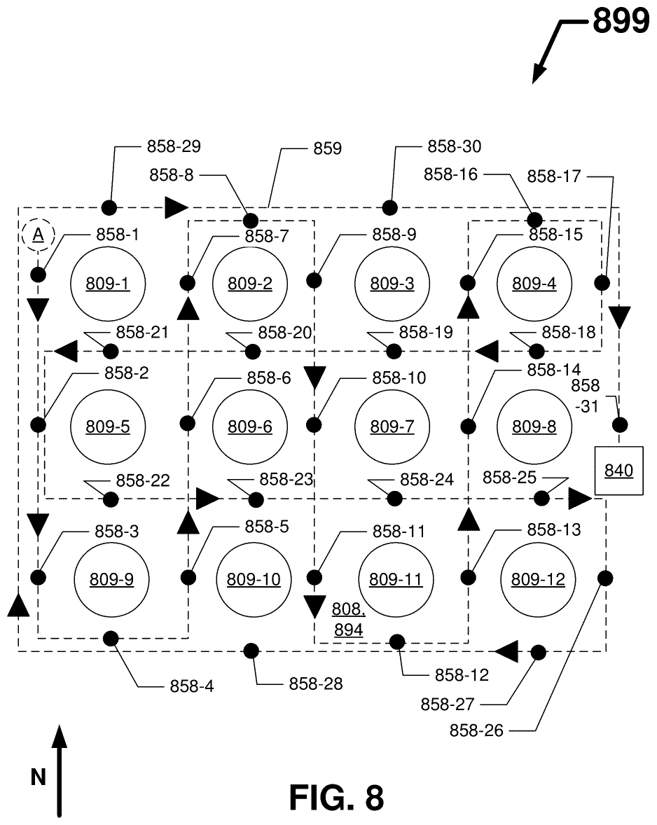

In certain example embodiments, the leak determination module 541 is further configured to generate and/or modify a path (such as the path 859 in FIG. 8 below and the path 959 in FIG. 9 below) and/or the sample points (such as the sample points 858 in FIG. 8 below and the sample points 958 in FIG. 9 below). Generating and/or modifying the path and/or the sample points may be based on one or more of a number of factors, including but not limited to the terrain, the number of storage vessels 409 (including the field equipment 411 thereof), the location of the storage vessels 409 (including the field equipment 411 thereof), the conditions in the outside environment 494, the measurements made by the sensor devices 460 of the mobile vehicle 440, the capability of the mobility features 495 of the mobile vehicle 440, the effectiveness of the processing apparatus 444 in converting the original samples 471 to the processed samples 472, the functionality of the sample collectors 445 and/or the sensor devices 460, the capabilities of the sensor devices 460, and the configuration of the testing apparatuses 470.

The path and/or the sample points may be generated and/or modified by a controller 404 (e.g., using one or more algorithms 533, one or more protocols 532, and/or stored data 534) and/or a user 451. The path and/or the sample points may be generated and/or modified in real time to improve science outcomes and the chance of correctly identifying a leak in a storage vessel 409. If the leak repair evaluation module 543 is used to help facilitate use of one or more repair features 443 in performing a repair of field equipment 411 in stopping or slowing a leak 401 thorough a leak point 412, the leak determination module 541 may alter a path of the mobile vehicle 440 so that measurements are taken in the vicinity of the leak point 412 to determine the effectiveness of the repairs.

The recommendation module 542 of the controller 404 may be configured to generate a recommendation regarding one or more of the storage vessels 409. For example, the recommendation module 542 may use stored data 534, one or more protocols 532, and/or one or more algorithms 533 to generate a recommendation as to whether a particular storage vessel 409 has a leak that needs to be repaired. In some cases, the recommendation module 542 may also make particular recommendations as to how (e.g., replace a particular valve) a leak in a storage vessel 409 may be repaired.

Implementation of the functions of the recommendation module 542 may be performed in one or more of a number of ways. For example, the recommendation module 542 may use stored data 534, one or more algorithms 533, and/or protocols 532 to determine that a difference between the measured results (e.g., as determined by the leak determination module 541) and the expected results (e.g., based on existing algorithms 533) exceeds a threshold forecast value. In such a case, the recommendation module 542 may use stored data 534, one or more algorithms 533, and/or protocols 532 to generate a revision to the algorithm 533 (e.g., a forecasting model) based on the difference. The recommendation module 542 may use stored data 534 that is derived from outputs of the leak determination module 541.

The leak repair evaluation module 543 of the controller 404 may be configured to evaluate a repair of a leak that has been made in a storage vessel 409. For example, the leak repair evaluation module 543 may use one or more protocols 532 and/or one or more algorithms 533, as well as stored data 534 (e.g., measurements of one or more parameters made by one or more sensor devices 460), to compare the results of testing samples proximate to a storage vessel 409 that had a leak that had been repaired at some point in the past to expected results generated by one or more algorithms 533 (e.g., a forecasting model). Any differences that exceed a threshold value may be used by the leak repair evaluation module 543 as a basis of evaluating whether the repair of a leak has been effective.

Implementation of the functions of the leak repair evaluation module 543 may be performed in one or more of a number of ways. For example, the leak repair evaluation module 543 may use stored data 534, one or more algorithms 533, and/or protocols 532 to provide an evaluation of a leak repair of a storage vessel 409 to the recommendation module 542, which may use this evaluation to generate a recommendation as to whether the repair of a leak should be reworked.

The communication module 507 of the controller 404 determines and implements the communication protocol (e.g., from the protocols 532 of the storage repository 531) that is used when the control engine 506 communicates with (e.g., sends signals to, obtains signals from) the user systems 455, the sample collectors 445, the mobility features 495, the processing apparatus 444, the sensor devices 460, the sensor devices 360, the controllers 304, the other controllers 404, the sample collectors 445, the mobility features 495, the processing apparatus 444, the network manager 480, and the other components of the system 400, including the example mobile vehicle 440. In some cases, the communication module 507 accesses the stored data 534 to determine which communication protocol is used to communicate with another component of the system 400. In addition, the communication module 507 may identify and/or interpret the communication protocol of a communication obtained by a controller 404 so that the control engine 506 may interpret the communication. The communication module 507 may also provide one or more of a number of other services with respect to data sent from and obtained by a controller 404. Such services may include, but are not limited to, data packet routing information and procedures to follow in the event of data interruption.

The timer 535 of a controller 404 may track clock time, intervals of time, an amount of time, and/or any other measure of time. The timer 535 may also count the number of occurrences of an event, whether with or without respect to time. Alternatively, the control engine 506 may perform a counting function. The timer 535 is able to track multiple time measurements and/or count multiple occurrences concurrently. The timer 535 may track time periods based on an Instruction obtained from the control engine 506, based on an instruction obtained from a user 451, based on an instruction programmed in the software for the controller 404, based on some other condition (e.g., the occurrence of an event) or from some other component, or from any combination thereof. In certain example embodiments, the timer 535 may provide a time stamp for each packet of data obtained from another component (e.g., a sensor device 460) of the system 400.

The power module 530 of a controller 404 obtains power from a power supply (e.g., AC mains) and manipulates (e.g., transforms, rectifies, inverts) that power to provide the manipulated power to one or more other components (e.g., the timer 535, the control engine 506) of the controller 404, where the manipulated power is of a type (e.g., alternating current, direct current) and level (e.g., 12V, 24V, 120V) that may be used by the other components of the controller 404. In some cases, the power module 530 may also provide power to one or more of the sensor devices 460.

The power module 530 may include one or more of a number of single or multiple discrete components (e.g., transistor, diode, resistor, transformer) and/or a microprocessor. The power module 530 may include a printed circuit board, upon which the microprocessor and/or one or more discrete components are positioned. In addition, or in the alternative, the power module 530 may be a source of power in itself to provide signals to the other components of the controller 404. For example, the power module 530 may be or include an energy storage device (e.g., a battery). As another example, the power module 530 may be or include a localized photovoltaic power system.

The hardware processor 521 of a controller 404 executes software, algorithms (e.g., algorithms 533), and firmware in accordance with one or more example embodiments. Specifically, the hardware processor 521 may execute software on the control engine 506 or any other portion of the controller 404, as well as software used by the users 451 (including associated user systems 455), the sample collectors 445, the mobility features 495, the processing apparatus 444, the network manager 480, and/or other components of the system 400. The hardware processor 521 may be an integrated circuit, a central processing unit, a multi-core processing chip, SoC, a multi-chip module including multiple multi-core processing chips, or other hardware processor in one or more example embodiments. The hardware processor 521 may be known by other names, including but not limited to a computer processor, a microprocessor, and a multi-core processor.

In one or more example embodiments, the hardware processor 521 executes software instructions stored in memory 522. The memory 522 includes one or more cache memories, main memory, and/or any other suitable type of memory. The memory 522 may include volatile and/or non-volatile memory. The memory 522 may be discretely located within the controller 404 relative to the hardware processor 521. In certain configurations, the memory 522 may be integrated with the hardware processor 521.

In certain example embodiments, the controller 404 does not include a hardware processor 521. In such a case, the controller 404 may include, as an example, one or more field programmable gate arrays (FPGA), one or more insulated-gate bipolar transistors (IGBTs), and/or one or more integrated circuits (ICs). Using FPGAs, IGBTs, ICs, and/or other similar devices known in the art allows the controller 404 (or portions thereof) to be programmable and function according to certain logic rules and thresholds without the use of a hardware processor. Alternatively, FPGAs, IGBTs, ICs, and/or similar devices may be used in conjunction with one or more hardware processors 521.

The transceiver 524 of a controller 404 may send and/or obtain control and/or communication signals. Specifically, the transceiver 524 may be used to transfer data between a controller 404 and the users 451 (including associated user systems 455), the sensor devices 460, the sensor devices 360, the controllers 304, the other controllers 404, the sample collectors 445, the mobility features 495, the processing apparatus 444, the network manager 480, and the other components of the system 400, including the example mobile vehicle 440. The transceiver 524 may use wired and/or wireless technology. The transceiver 524 may be configured in such a way that the control and/or communication signals sent and/or obtained by the transceiver 524 may be obtained and/or sent by another transceiver that is part of a user system 455, a sensor device 460, the sensor devices 360, the controllers 304, the other controllers 404, the sample collectors 445, the mobility features 495, the processing apparatus 444, the network manager 480, and/or another component of the system 400, including the example mobile vehicle 440. The transceiver 524 may send and/or obtain any of a number of signal types, including but not limited to radio frequency signals.

When the transceiver 524 uses wireless technology, any type of wireless technology may be used by the transceiver 524 in sending and obtaining signals. Such wireless technology may include, but is not limited to, Wi-Fi, Zigbee, VLC, cellular networking, BLE, UWB, and Bluetooth. The transceiver 524 may use one or more of any number of suitable communication protocols (e.g., ISA100, HART) when sending and/or obtaining signals. The transceiver 524 may send and receive the communication signals using one or more of the communication links 405.

Optionally, in one or more example embodiments, the security module 523 secures interactions between a controller 404, the users 451 (including associated user systems 455), the sensor devices 460, the sensor devices 360, the controllers 304, the other controllers 404, the sample collectors 445, the mobility features 495, the processing apparatus 444, the network manager 480, and the other components of the system 400, including the example mobile vehicle 440. More specifically, the security module 523 authenticates communication from software based on security keys verifying the identity of the source of the communication. For example, user software may be associated with a security key enabling the software of a user system 455 to interact with a controller 404. Further, the security module 523 may restrict receipt of information, requests for information, and/or access to information.

A user 451 (which may include an associated user system 455), the sensor devices 460, the sensor devices 360, the controllers 304, the other controllers 404, the sample collectors 445, the mobility features 495, the processing apparatus 444, the network manager 480, and the other components of the system 400 may interact with a controller 404 using the application interface 526. Specifically, the application interface 526 of a controller 404 obtains data (e.g., information, communications, instructions, updates to firmware) from and sends data (e.g., information, communications, instructions) to the user systems 455 of the users 451, the sensor devices 460, the sensor devices 360, the controllers 304, the other controllers 404, the sample collectors 445, the mobility features 495, the processing apparatus 444, the network manager 480, and/or the other components of the system 400.

Examples of an application interface 526 may be or include, but are not limited to, an application programming interface, a web service, a data protocol adapter, some other hardware and/or software, or any suitable combination thereof. Similarly, the user systems 455 of the users 451, the sensor devices 460, the sensor devices 360, the controllers 304, the other controllers 404, the sample collectors 445, the mobility features 495, the processing apparatus 444, the network manager 480, and/or the other components of the system 400 may include an interface (similar to the application interface 526 of the controller 404) to obtain data from and send data to a controller 404 in certain example embodiments.

In addition, as discussed above with respect to a user system 455 of a user 451, one or more of the sensor devices 460, one or more of the sensor devices 360, one or more of the controllers 304, one or more of the other controllers 404, one or more of the sample collectors 445, one or more of the mobility features 495, some or all of the processing apparatus 444, the network manager 480, and/or one or more of the other components (or portions thereof) of the system 400 may include a user interface. Examples of such a user interface may include, but are not limited to, a graphical user interface, a touchscreen, a keyboard, a monitor, a mouse, some other hardware, or any suitable combination thereof.

The controllers 404, the users 451 (including associated user systems 455), the sensor devices 460, the sensor devices 360, the controllers 304, the sample collectors 445, the mobility features 495, the processing apparatus 444, the network manager 480, and the other components of the system 400 may use their own system or share a system in certain example embodiments. Such a system may be, or contain a form of, an Internet-based or an intranet-based computer system that is capable of communicating with various software. A computer system includes any type of computing device and/or communication device, including but not limited to a controller 404. Examples of such a system may include, but are not limited to, a desktop computer with a Local Area Network (LAN), a Wide Area Network (WAN), Internet or intranet access, a laptop computer with LAN, WAN, Internet or intranet access, a smart phone, a server, a server farm, an android device (or equivalent), a tablet, smartphones, and a personal digital assistant (PDA). Such a system may correspond to a computer system as described below with regard to FIG. 6.

Further, as discussed above, such a system may have corresponding software (e.g., user system software, sensor device software, controller software). The software may execute on the same or a separate device (e.g., a server, mainframe, desktop personal computer (PC), laptop, PDA, television, cable box, satellite box, kiosk, telephone, mobile phone, or other computing devices) and may be coupled by the communication network (e.g., Internet, Intranet, Extranet, LAN, WAN, or other network communication methods) and/or communication channels, with wire and/or wireless segments according to some example embodiments. The software of one system may be a part of, or operate separately but in conjunction with, the software of another system within the system 400.

FIG. 6 illustrates one embodiment of a computing device 618 that implements one or more of the various techniques described herein, and which is representative, in whole or in part, of the elements described herein pursuant to certain example embodiments. For example, a controller 404 (including components thereof, such as a control engine 506, a hardware processor 521, a storage repository 531, a power module 530, and a transceiver 524) may be considered a computing device 618 (also called a computer system herein). Computing device 618 is one example of a computing device and is not intended to suggest any limitation as to scope of use or functionality of the computing device and/or its possible architectures. Neither should the computing device 618 be interpreted as having any dependency or requirement relating to any one or combination of components illustrated in the example computing device 618.

The computing device 618 includes one or more processors or processing units 614, one or more memory/storage components 615, one or more input/output (I/O) devices 616, and a bus 617 that allows the various components and devices to communicate with one another. The bus 617 represents one or more of any of several types of bus structures, including a memory bus or memory controller, a peripheral bus, an accelerated graphics port, and a processor or local bus using any of a variety of bus architectures. The bus 617 includes wired and/or wireless buses.

The memory/storage component 615 represents one or more computer storage media. The memory/storage component 615 includes volatile media (such as random access memory (RAM)) and/or nonvolatile media (such as read only memory (ROM), flash memory, optical disks, magnetic disks, and so forth). The memory/storage component 615 includes fixed media (e.g., RAM, ROM, a fixed hard drive, etc.) as well as removable media (e.g., a Flash memory drive, a removable hard drive, an optical disk, and so forth).

One or more I/O devices 616 allow a user 451 to enter commands and information to the computing device 618, and also allow information to be presented to the user 451 and/or other components or devices. Examples of input devices 616 include, but are not limited to, a keyboard, a cursor control device (e.g., a mouse), a microphone, a touchscreen, and a scanner. Examples of output devices include, but are not limited to, a display device (e.g., a monitor or projector), speakers, outputs to a lighting network (e.g., DMX card), a printer, and a network card.

Various techniques are described herein in the general context of software or program modules. Generally, software includes routines, programs, objects, components, data structures, and so forth that perform particular tasks or implement particular abstract data types. An implementation of these modules and techniques are stored on or transmitted across some form of computer readable media. Computer readable media is any available non-transitory medium or non-transitory media that is accessible by a computing device. By way of example, and not limitation, computer readable media includes “computer storage media”.

“Computer storage media” and “computer readable medium” include volatile and non-volatile, removable and non-removable media implemented in any method or technology for storage of information such as computer readable instructions, data structures, program modules, or other data. Computer storage media include, but are not limited to, computer recordable media such as RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which is used to store the desired information and which is accessible by a computer.

The computer device 618 is connected to a network (not shown) (e.g., a LAN, a WAN such as the Internet, cloud, or any other similar type of network) via a network interface connection (not shown) according to some example embodiments. Those skilled in the art will appreciate that many different types of computer systems exist (e.g., desktop computer, a laptop computer, a personal media device, a mobile device, such as a cell phone or personal digital assistant, or any other computing system capable of executing computer readable instructions), and the aforementioned input and output means take other forms, now known or later developed, in other example embodiments. Generally speaking, the computer device 618 includes at least the minimal processing, input, and/or output means necessary to practice one or more embodiments.

Further, those skilled in the art will appreciate that one or more elements of the aforementioned computer device 618 is located at a remote location and connected to the other elements over a network in certain example embodiments. Further, one or more embodiments is implemented on a distributed system having one or more nodes, where each portion of the implementation (e.g., a sample collector 445, a testing apparatus 470, the processing apparatus 444) is located on a different node within the distributed system. In one or more embodiments, the node corresponds to a computer system. Alternatively, the node corresponds to a processor with associated physical memory in some example embodiments. The node alternatively corresponds to a processor with shared memory and/or resources in some example embodiments.

FIG. 7 shows a flowchart 758 of a method for detecting a leak of a gas 485 from a vessel 409 in an outdoor environment 494 according to certain example embodiments. While the various steps in this flowchart 758 are presented sequentially, one of ordinary skill will appreciate that some or all of the steps may be executed in different orders, may be combined or omitted, and some or all of the steps may be executed in parallel. Further, in one or more of the example embodiments, one or more of the steps shown in this example method may be omitted, repeated, and/or performed in a different order. Some or all of the steps of the method of FIG. 7 may be performed off site (e.g., in a laboratory remote from a field operation). In addition, or in the alternative, some or all of the steps of the method of FIG. 7 may be performed on site (e.g., in the outside environment 494, adjacent to a storage vessel 409 (including associated field equipment 411)) where potential leaks are being detected by an example mobile vehicle 440.

In addition, a person of ordinary skill in the art will appreciate that additional steps not shown in FIG. 7 may be included in performing this method. Accordingly, the specific arrangement of steps should not be construed as limiting the scope. Further, a particular computing device, such as the computing device 618 discussed above with respect to FIG. 6, may be used to facilitate (e.g., direct, control, provide instructions, provide recommendations, perform, execute) performance of one or more of the steps for the methods shown in FIG. 7 in certain example embodiments. Any of the functions performed below by a controller 404 (an example of which is shown in FIG. 5) may involve the use of one or more protocols 532, one or more algorithms 533, and/or stored data 534 stored in a storage repository 531. In addition, or in the alternative, any of the functions (or portions thereof) in the method may be performed by a user (e.g., user 451).

The method shown in FIG. 7 is merely an example that may be performed by using an example system described herein. In other words, systems for detecting a leak of a gas 485 from a vessel 409 in an outdoor environment 494 may perform other functions using other methods in addition to and/or aside from those described with respect to FIG. 7. Incorporating the description above with respect to FIGS. 1 through 6, the method shown in the flowchart 758 of FIG. 7 begins at the START step and proceeds to step 781, where an original sample 471 is obtained from the outside environment 494. As used herein, the term “obtaining” may include collecting, receiving, retrieving, accessing, generating, etc. or any other manner of obtaining original samples 471 from the outside environment 494.

Each original sample 471 may be obtained by an example mobile vehicle 440 using one or more of the sample collectors 445. Each original sample 471 is obtained from the outside environment 494 at a location that may or may not be adjacent to a storage vessel 409 (including associated field equipment 411). Each original sample 471 may be obtained while the mobile vehicle 440 is traveling along a path (e.g., such as the path 959 of FIG. 9 below).

Some or all of the process of obtaining the original samples 471 may be controlled by a controller 404 (or a collecting component thereof) of the analytic system 450 or other part of the mobile vehicle 440 using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of obtaining the original samples 471 may be controlled by a user 451. The original samples 471 may be obtained from the outside environment 494 continuously over an extended period of time or on an iterative basis. The rate (e.g., hourly, daily, weekly, randomly) of collecting the original samples 471 may vary (e.g., based on terrain, based on field conditions, based on the path of the mobile vehicle 440, based on whether there is no significant change in the various measured properties of original samples 471 obtained from adjacent locations) over time. Original samples 471 may be obtained from different heights relative to the ground (e.g., ground 308).

In step 782, the original sample 471 is processed to generate a processed sample 472. The original sample 471 may be processed by the processing apparatus 444 of the mobile vehicle 440. The original sample 471 may be processed using any of a number of implementations, including but not limited to filtering, mixing, compressing, cooling, heating, agitating, separating, and depressurizing. For example, the processed sample 472 may be generated by removing water vapor from the original sample 471. As another example, the processed sample 472 may be generated by removing a quantity of the gas 485 that naturally occurs in the outside environment 494 from the original sample 471.

Some or all of the process of processing the original sample 471 to generate the processed sample 472 may be controlled by a controller 404 (or the leak determination module 541 thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of processing the original sample 471 to generate the processed sample 472 may be controlled by a user 451. The original sample 471 may be processed continuously over an extended period of time or on a discrete basis. The original sample 471 may be processed in a single stage or in multiple stages. In certain example embodiments, the original sample 471 is processed in real time (e.g., at substantially the same time as when the original sample 471 is obtained).

In step 783, measurements of one or more parameters associated with a gas 485 in the processed sample 472 are obtained. Measurements of the one or more parameters associated with a gas 485 in the processed sample 472 may be made, directly or indirectly, using one or more sensor devices 460. The parameters associated with a gas 485 in the processed sample 472 may be measured in one or more testing vessels 473 of the testing apparatuses 470 of the analytic system 450. The parameters associated with a gas 485 in the processed sample 472 may include, but are not limited to, the amount of the gas 485 and the temperature of the gas 485.

In some cases, measurements of one or more parameters associated with the processed sample 472, the original sample 471, the mobile vehicle 440, and/or the outside environment 494 are obtained. Measurements of the one or more parameters associated with the processed sample 472, the original sample 471, the mobile vehicle 440, and/or the outside environment 494 may be made, directly or indirectly, using one or more sensor devices 460. The parameters associated with a gas 485 in the processed sample 472 may include, but are not limited to, wind speed, wind direction, temperature of the outside environment 494, humidity, composition of the processed sample 472, composition of the original sample 471, and the GPS coordinates of the mobile vehicle 440.

Some or all of the process of obtaining measurements of the parameters associated with a gas 485 in the processed sample 472, the rest of processed sample 472, the original sample 471, the mobile vehicle 440, and/or the outside environment 494 may be controlled by a controller 404 (or the leak determination module 541 thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of obtaining measurements of the parameters associated with a gas 485 in the processed sample 472, the rest of processed sample 472, the original sample 471, the mobile vehicle 440, and/or the outside environment 494 may be controlled by a user 451. The parameters associated with a gas 485 in the processed sample 472, the rest of processed sample 472, the original sample 471, the mobile vehicle 440, and/or the outside environment 494 may be measured continuously over an extended period of time or on a discrete basis. In certain example embodiments, the measurements of the parameters are made by and obtained from the sensor devices 460 in real time (e.g., at substantially the same time as when the processed sample 472 is generated).

In step 784, the measurements obtained in step 783 are compared with a range of acceptable values. A comparison of the measurements of the parameters associated with the gas 485 may be made by a controller 404 (or the leak determination module 541)) of the analytic system 450 of the mobile vehicle 440 using one or more protocols 532, one or more algorithms 533 (e.g., models), and stored data 534. The comparisons may result in or contribute to a trend, the establishment of or modification to a baseline and/or the range of acceptable values, the creation or modification of an algorithm 533, and/or some other predictive indication (e.g., identification of a leak 401) associated with the gas 485. In certain example embodiments, this step 784 may be used to establish a calibration or reference database to help forecast and/or improve detection of leaks 401 in storage vessels 409 (including associated field equipment 411).

Some or all of the process of comparing the measurements of the parameters associated with a gas 485 in the processed sample 472 with a range of acceptable values may be controlled by a controller 404 (or the leak determination module 541 thereof) using one or more protocols 532, one or more algorithms 533 (e.g., models), measurements of one or more sensor devices 460, input from a user 451 (which may include an associated user system 455), and/or any other source of information within the system 400. In addition, or in the alternative, some or all of the process of comparing the measurements of the parameters associated with a gas 485 in the processed sample 472 with a range of acceptable values may be controlled by a user 451. The measurements of the parameters associated with a gas 485 in the processed sample 472 with a range of acceptable values may be compared continuously over an extended period of time or on a discrete basis. In certain example embodiments, the measurements of the parameters associated with a gas 485 in the processed sample 472 may be compared with a range of acceptable values in real time (e.g., at substantially the same time as when the measurements are obtained from the sensor devices 460).

In step 786, a determination is made as to whether the measurement falls within the range of acceptable values. Such a determination may be based on whether any of a number of factors, including but not limited to the number of measurements (e.g., consecutive measurements) that fall outside the range of acceptable values, whether external factors (e.g., exhaust from the mobile vehicle 440) survived the processing of the original sample 471, the amount that the measurements fall outside the range of acceptable values, historical readings at that location in the outside environment 494, the values of measurements made from adjacent locations in the outside environment 494, and whether the range of acceptable values are current.

The determination may be made by a controller 404 (or the leak determination module 541 thereof) of the analytic system 450 of the mobile vehicle 440 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, the determination may be made by a user 451. In certain example embodiments, the determination is made in real time (e.g., at substantially the same time as when the measurements are compared with the range of acceptable values). If the measurement falls within the range of acceptable values, then the process proceeds to step 787. If the measurement falls within the range of acceptable values, then the process proceeds to step 761.

In step 787, a determination is made as to whether another original sample 471 is collected at the location in the outside environment 494. Such a determination may be based on whether any of a number of factors, including but not limited to whether additional data is needed to conclude that a leak 401 of the gas 485 exists at or near the location in the outside environment 494, the amount of time that has lapsed since the prior original sample 471 was obtained, external conditions (e.g., wind speed, wind direction) in the outside environment 494, where within the range of acceptable values the measurement of the prior processed sample 472 falls, and the number of original samples 471 already taken at the location in the outside environment 494.

The determination may be made by a controller 404 (or the leak determination module 541 thereof) of the analytic system 450 of the mobile vehicle 440 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, the determination may be made by a user 451. In certain example embodiments, the determination is made in real time (e.g., at substantially the same time as when the determination that the prior measurement falls within the range of acceptable values is made). If another original sample 471 is obtained at the location in the outside environment 494, then the process reverts to step 781. If another original sample 471 is obtained at the location in the outside environment 494, then the process proceeds to step 788.

In step 788, a determination is made as to whether another original sample 471 is collected at a different location in the outside environment 494. Such a determination may be based on whether any of a number of factors, including but not limited to whether additional data is needed to conclude that a leak 401 of the gas 485 exists at or near the location in the outside environment 494, whether the different location of the outside environment 494 has yet been evaluated, the amount of time that has lapsed since the prior original sample 471 was obtained, external conditions (e.g., wind speed, wind direction) in the outside environment 494, where within the range of acceptable values the measurement of the prior processed sample 472 falls, and the number of original samples 471 already taken at the location in the outside environment 494.

The determination may be made by a controller 404 (or the leak determination module 541 thereof) of the analytic system 450 of the mobile vehicle 440 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, the determination may be made by a user 451. In certain example embodiments, the determination is made in real time (e.g., at substantially the same time as when the determination as to whether the prior measurement falls within the range of acceptable values). If another original sample 471 is obtained at a different location in the outside environment 494, then the process proceeds to step 789. If another original sample 471 is obtained at a different location in the outside environment 494, then the process proceeds to the END step.

In step 789, the mobile vehicle 440 is moved. Specifically, the mobile vehicle 440 is moved to a different location in the outside environment 494. The mobile vehicle 440 may be moved using one or more of the mobility features 495 of the mobile vehicle 440. Moving the mobile vehicle 440, including determining the new location in the outside environment 494, may be controlled by a controller 404 (or the leak determination module 541 thereof) of the analytic system 450 of the mobile vehicle 440 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, moving the mobile vehicle 440 may be controlled by a user 451. In certain example embodiments, the mobile vehicle 440 is moved in real time (e.g., at substantially the same time as when the determination that another original sample 471 is to be taken at another location in the outside environment 494). When step 789 is completed, the process reverts to step 781.

In step 761, the characteristics of the leak 401 of the gas 485 are identified. The characteristics of the leak 401 of the gas 485 may include, but are not limited to, one or more of the following: The extent of the leak 401 (e.g., the quantity of the gas 485 measured), the location in the outside environment 494 of the leak 401, the composition of the gas 485, the one or more storage vessels 409 from which the gas 485 has leaked, the leak point 412 of each storage vessel 409, and the failure 413 in the field equipment 411 of the storage vessel 409.

One or more characteristics of the leak 401 of the gas 485 may be identified by a controller 404 (or the leak determination module 541 and/or the leak repair evaluation module 543 thereof) of the analytic system 450 of the mobile vehicle 440 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, one or more characteristics of the leak 401 of the gas 485 may be identified by a user 451. In certain example embodiments, one or more characteristics of the leak 401 of the gas 485 may be identified in real time (e.g., at substantially the same time as when the determination that the prior measurement falls outside the range of acceptable values is made). If the mobile vehicle 440 has repair capabilities (e.g., among the repair features 443), then the process proceeds to optional step 762. If the mobile vehicle 440 does not have repair capabilities, then the process proceeds to optional step 763.

In step 762, fixing the leak 401 of the gas 485 is facilitated. Fixing the leak 401 may be eliminating the leak 401 or reducing the extent of the leak 401. Fixing the leak 401 may be a permanent repair or a temporary repair of the leak point 412. Fixing the leak 401 of the gas 485 may be facilitated by the use of one or more of the optional repair features 443 on the mobile vehicle 440. One or more of the repair features 443 may be selected and/or controlled by a controller 404 (or the leak repair evaluation module 543 thereof) of the analytic system 450 of the mobile vehicle 440 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, one or more of the repair features 443 may be selected and/or controlled by a user 451. In certain example embodiments, fixing the leak 401 of the gas 485 may be facilitated in real time (e.g., at substantially the same time as when the characteristics of the leak 401 of the gas 485 are identified). When optional step 762 is completed, the process proceeds to step 763.

In step 763, the leak 401 of the gas 485 is reported. The leak 401 of the gas 485 may be reported to a user 451 (including an associated user system 455), the network manager 480, and/or some other entity within the system 400. Reporting the leak 401 of the gas 485 may including any relevant information, including but not limited to the location in the outside environment 494 where the leak 401 is detected, the day/time that the leak 401 is detected, the content of the gas 485 of the leak 401, the storage vessel 409 from which the leak 401 originates, the suspected leak point 412 and/or failure 413 of the storage vessel 409 (including any associated field equipment 411), any corrective actions that have been taken to fix the leak 401, recommendations as to corrective actions to take to fix the leak 401, and the size of the leak 401.

The leak 401 of the gas 485 may be reported by a controller 404 (or the leak determination module 541, the recommendation module 542, and/or the leak repair evaluation module 543 thereof) of the analytic system 450 of the mobile vehicle 440 using one or more protocols 532, one or more algorithms 533 (e.g., models), stored data 534, measurements of one or more sensor devices 460, input from a user 451 (which may include an associated user system 455), and/or any other source of information within the system 400. In addition, or in the alternative, the leak 401 of the gas 485 may be reported by a user 451. In certain example embodiments, the leak 401 of the gas 485 may be reported in real time (e.g., at substantially the same time as when the characteristics of the leak 401 of the gas 485 are identified). When step 763 is completed, the process proceeds to the END step.

FIG. 8 shows a top view of a system 899 in which an example mobile vehicle 840 tests for leaks around 12 storage vessels 809 in accordance with certain example embodiments. Referring to the description above with respect to FIGS. 1 through 7, the system 899 of FIG. 8 includes the 12 storage vessels 809 (storage vessel 809-1, storage vessel 809-2, storage vessel 809-3, storage vessel 809-4, storage vessel 809-5, storage vessel 809-6, storage vessel 809-7, storage vessel 809-8, storage vessel 809-9, storage vessel 809-10, storage vessel 809-11, and storage vessel 809-12) arranged substantially equidistantly in an outside environment 894 in 3 rows and 4 columns. Each storage vessel 809 stores a gas (similar to the gases (e.g., gas 485) discussed above).

The mobile vehicle 840 (e.g., a truck, a UAV) travels along a path 859 (e.g., on the ground 808, above the ground 808) in the outside environment 894 to obtain multiple original samples (similar to the original samples (e.g., original samples 471) discussed above). The path 859 may be generated by a controller (e.g., controller 404) of the mobile vehicle 840 and/or a user (e.g., user 451). The path 859 starts at point A and weaves around and between all of the storage vessels 809 at least once. In its travel along the path 859, the mobile vehicle 840 obtains original samples at 31 different sample points 858. In alternative embodiments, the mobile vehicle 840 obtains original samples continuously along the path 859. The mobile vehicle 840 and its various features (e.g., the mobility features 495, the testing apparatuses 470, the sample collectors 445) may be operated and/or controlled by a user (e.g., user 451) and/or a controller (e.g., controller 404).

An original sample is obtained at sample point 858-1 adjacent to the west of storage vessel 809-1. Subsequently, another original sample is obtained at sample point 858-2 adjacent to the west of storage vessel 809-5. Subsequently, another original sample is obtained at sample point 858-3 adjacent to the west of storage vessel 809-9. Subsequently, another original sample is obtained at sample point 858-4 adjacent to the south of storage vessel 809-9. Subsequently, another original sample is obtained at sample point 858-5 between storage vessel 809-9 and storage vessel 809-10. Subsequently, another original sample is obtained at sample point 858-6 between storage vessel 809-5 and storage vessel 809-6. Subsequently, another original sample is obtained at sample point 858-7 between storage vessel 809-1 and storage vessel 809-2. Subsequently, another original sample is obtained at sample point 858-8 adjacent to the north of storage vessel 809-2. Subsequently, another original sample is obtained at sample point 858-9 between storage vessel 809-2 and storage vessel 809-3.

Subsequently, another original sample is obtained at sample point 858-10 between storage vessel 809-6 and storage vessel 809-7. Subsequently, another original sample is obtained at sample point 858-11 between storage vessel 809-10 and storage vessel 809-11. Subsequently, another original sample is obtained at sample point 858-12 adjacent to the south of storage vessel 809-11. Subsequently, another original sample is obtained at sample point 858-13 between storage vessel 809-11 and storage vessel 809-12. Subsequently, another original sample is obtained at sample point 858-14 between storage vessel 809-7 and storage vessel 809-8. Subsequently, another original sample is obtained at sample point 858-15 between storage vessel 809-3 and storage vessel 809-4. Subsequently, another original sample is obtained at sample point 858-16 adjacent to the north of storage vessel 809-4. Subsequently, another original sample is obtained at sample point 858-17 adjacent to the east of storage vessel 809-4.

Subsequently, another original sample is obtained at sample point 858-18 between storage vessel 809-4 and storage vessel 809-8. Subsequently, another original sample is obtained at sample point 858-19 between storage vessel 809-3 and storage vessel 809-7. Subsequently, another original sample is obtained at sample point 858-20 between storage vessel 809-2 and storage vessel 809-6. Subsequently, another original sample is obtained at sample point 858-21 between storage vessel 809-1 and storage vessel 809-5. Subsequently, another original sample is obtained at sample point 858-22 between storage vessel 809-5 and storage vessel 809-9. Subsequently, another original sample is obtained at sample point 858-23 between storage vessel 809-6 and storage vessel 809-10. Subsequently, another original sample is obtained at sample point 858-24 between storage vessel 809-7 and storage vessel 809-11. Subsequently, another original sample is obtained at sample point 858-25 between storage vessel 809-8 and storage vessel 809-12.

Subsequently, another original sample is obtained at sample point 858-26 adjacent to the east of storage vessel 809-12. Subsequently, another original sample is obtained at sample point 858-27 adjacent to the south of storage vessel 809-12. Subsequently, another original sample is obtained at sample point 858-28 adjacent to the south of storage vessel 809-10. Subsequently, another original sample is obtained at sample point 858-29 adjacent to the north of storage vessel 809-1. Subsequently, another original sample is obtained at sample point 858-30 adjacent to the north of storage vessel 809-3. Subsequently, another original sample is obtained at sample point 858-31 adjacent to the east of storage vessel 809-8.

By processing each of the original samples (similar to the original samples 471 discussed above) taken from the various sample points 858 in the outside environment 894, processing each sample, testing each processed sample (similar to the processed samples 472 discussed above), and comparing the results of the tests to acceptable values, a controller of the mobile vehicle 840 can determine, in real time, whether any of the storage vessels 809 has a leak that is releasing a gas into the outside environment 894.

In some cases, when a controller of the mobile vehicle 840 determines that one or more of the storage vessels 809 has a leak that is releasing a gas into the outside environment 894, the controller of the mobile vehicle 840 may determine which of the storage vessels 809 has a leak. Such a determination may incorporate the use of measurements of parameters (e.g., wind speed, wind direction, humidity) made by one or more sensor devices (e.g., similar to the sensor devices 460 discussed above) of the mobile vehicle 840 and/or other information (e.g., the configuration of each storage vessel 809, the contents of the gas contained within each storage vessel 809) available (e.g., as stored data 534) to a controller of the mobile vehicle 840. A controller of the mobile vehicle 840 may also determine, in some cases, where the leak on a storage vessel 809 is and how the leak may be most effectively stopped or reduced. In this example, none of the storage vessels 809 has a leak.

FIG. 9 shows a top view of another system 999 in which an example mobile vehicle 940 tests for leaks around the field equipment 911 for 4 storage vessels in accordance with certain example embodiments. Referring to the description above with respect to FIGS. 1 through 8, the storage vessels (formed by part of the subterranean formation (e.g., the subterranean formation 110), similar to the storage vessels 109 discussed above) are each capped with the field equipment 111 (field equipment 111-1, field equipment 111-2, field equipment 111-3, and field equipment 111-4) arranged randomly in an outside environment 994 among three hills 919 (hill 919-1, hill 919-2, and hill 919-3). In this case, each field equipment 111 covers the entry point of an injection well that terminates in part of the subterranean formation used as a storage vessel. Each storage vessel 909 stores a gas (similar to the gases (e.g., gas 485) discussed above).

The mobile vehicle 940 (e.g., a truck, a UAV) travels along a path 959 (e.g., on the ground 908, above the ground 908) in the outside environment 994 to obtain multiple original samples (similar to the original samples (e.g., original samples 471) discussed above). The path 959 may be generated by a controller (e.g., controller 404) of the mobile vehicle 940 and/or a user (e.g., user 451). The path 959 starts at point A and encircles each of the field equipment 911 once. In its travel along the path 959, the mobile vehicle 940 obtains original samples at 17 different sample points 958. In alternative embodiments, the mobile vehicle 940 obtains original samples continuously along the path 959. The mobile vehicle 940 and its various features (e.g., the mobility features 495, the testing apparatuses 470, the sample collectors 445) may be operated and/or controlled by a user (e.g., user 451) and/or a controller (e.g., controller 404).

An original sample is obtained at sample point 958-1 adjacent to the west of field equipment 911-1. Subsequently, another original sample is obtained at sample point 958-2 adjacent to the south of field equipment 911-1. Subsequently, another original sample is obtained at sample point 958-3 adjacent to the east of field equipment 911-1. Subsequently, another original sample is obtained at sample point 958-4 adjacent to the north of field equipment 911-1. Subsequently, after traveling from field equipment 911-1 to field equipment 911-2, another original sample is obtained at sample point 958-5 adjacent to the north of field equipment 911-2. Subsequently, another original sample is obtained at sample point 958-6 adjacent to the east of field equipment 911-2. Subsequently, another original sample is obtained at sample point 958-7 adjacent to the south of field equipment 911-2. Subsequently, another original sample is obtained at sample point 958-8 adjacent to the west of field equipment 911-2.

Subsequently, after traveling from field equipment 911-2 to field equipment 911-3, another original sample is obtained at sample point 958-9 adjacent to the northwest of field equipment 911-3. Subsequently, another original sample is obtained at sample point 958-10 adjacent to the northeast of field equipment 911-3. Subsequently, another original sample is obtained at sample point 958-11 adjacent to the southeast of field equipment 911-3. Subsequently, another original sample is obtained at sample point 958-12 adjacent to the southwest of field equipment 911-3.

On the way to traveling from field equipment 911-3 to field equipment 911-4, the mobile vehicle 940 obtains another original sample at sample point 958-13 adjacent to the northeast of field equipment 911-1. This is an example of when the leak determination module (e.g., leak determination module 541) of a controller (e.g., controller 404) of the mobile vehicle 940 may modify the path 959 and/or the sampling points 958 in real time to help determine whether the field equipment 911-3 of storage vessel 909-3 has a leak point 912, the location of the leak point 912, and/or the cause of the leak point 912. In alternative embodiments, the leak determination module of a controller of the mobile vehicle 940 may modify the path 959 to have the mobile vehicle 940 circle around each storage vessel 909 multiple times (e.g., in the same direction, in opposite directions), change the location and/or number of sample points 958 along the way, etc., based on real-time data (e.g., measurements of the processed samples and the corresponding location in the outside environment 994 of each corresponding original sample).

Subsequently, after completing travel from field equipment 911-3 to field equipment 911-4, another original sample is obtained at sample point 958-14 adjacent to the north of field equipment 911-4. Subsequently, another original sample is obtained at sample point 958-15 adjacent to the east of field equipment 911-4. Subsequently, another original sample is obtained at sample point 958-16 adjacent to the south of field equipment 911-4. Subsequently, another original sample is obtained at sample point 958-17 adjacent to the west of field equipment 911-4.

By processing each of the original samples (similar to the original samples 471 discussed above) taken from the various sample points 958 in the outside environment 994, processing each sample, testing each processed sample (similar to the processed samples 472 discussed above), and comparing the results of the tests to acceptable values, a controller of the mobile vehicle 940 can determine, in real time, whether any of the storage vessels 909 has a leak that is releasing a gas into the outside environment 994. In this example, a controller of the mobile vehicle 940 has determined that there is failure 913 in the field equipment 911-1, resulting in a leak point 912 (also sometimes called a source of the leak 901) through which a leak 901 of gas 985 stored in the storage vessel associated with the field equipment 911-1 escapes into the outside environment 994.

To make this determination, a controller of the mobile vehicle 940 may incorporate measurements of parameters (e.g., wind speed, wind direction, humidity) made by one or more sensor devices (e.g., similar to the sensor devices 460 discussed above) of the mobile vehicle 940 and/or other information (e.g., the configuration of each storage vessel 909, the contents of the gas contained within each storage vessel 909) available (e.g., as stored data 534) to a controller of the mobile vehicle 940. A controller of the mobile vehicle 940 may also determine, in some cases, where the leak point 912 in the field equipment 911-1 is and how the leak 901 may be most effectively stopped or reduced.

In certain example embodiments, for emissions derivation from the mobile vehicle 940, repeat transect data may be collected orthogonal to the winds. In some cases, road orientation, topography, and access generally prevent the collection of ideal (e.g., free from emissions from the mobile vehicle 940) data from the processed samples. In this way, non-orthogonal and/or arcing transects through plumes may be corrected by a controller (e.g., controller 404) in the data analysis. For example, an alternative approach to collecting plume data may be to slowly and repeatedly circle storage vessels 909 to collect original samples (e.g., plume data) in a series of arcs around each storage vessel 909.

Exhaust contamination from the mobile vehicle 940 having a combustion engine may occur in one or more of a number of situations. For example, when the mobile vehicle 940 moves slowly downwind at near the wind speed, has rapid deceleration, and/or reverses direction and crosses the exhaust plume downwind, exhaust contamination of the original samples may occur. A controller of the mobile vehicle 940 may use stored data (e.g., similar to the stored data 534 discussed above) in the form of analyzer data series, along with one or more algorithms and/or one or more protocols, to identify and flag exhaust-contaminated data that result from testing the original samples and/or the processed samples.

In some cases, a survey strategy (e.g., the path 959, the location of the sample points 958 along the path 959) may ensure that non-contaminated data are collected, particularly given the constraints imposed by roads, infrastructure, topography, etc., which can greatly complicate wind fields. For example, surveys may be conducted in opposite directions relative to the vehicle-wind orientation, and a controller (e.g., controller 404) of the mobile vehicle 940 may uses the measurements made by the sensor devices (e.g., sensor devices 460), along with one or more algorithms, one or more protocols, and/or stored data, to filter out the contaminated data. Additionally, or in the alternative, rapid deceleration of the mobile vehicle 940 may be avoided by having the mobile vehicle 940 move slower along its path 959. Slower speeds of the mobile vehicle 940 may also be useful for safety reasons, traffic control, reduced visibility, and/or repeat data collection from a sample point 958 (e.g., because of a faulty original sample, because of a suspected faulty reading by a sensor device).

Exhaust contamination may be quality flagged based on one or more factors. One factor is carbon monoxide (CO), which is a strong indicator of combustion, particularly when combined with elevated carbon dioxide (CO₂). Another factor is wind direction aligned with the mobile vehicle 940. Another factor is black carbon, which indicates diesel combustion and may indicate contamination from the exhaust of another vehicle or motor that operates nearby in the outside environment 994. Other factors may include aerosol concentration, size distribution, water (e.g., water vapor), and trace gases like nitric oxide (NO).

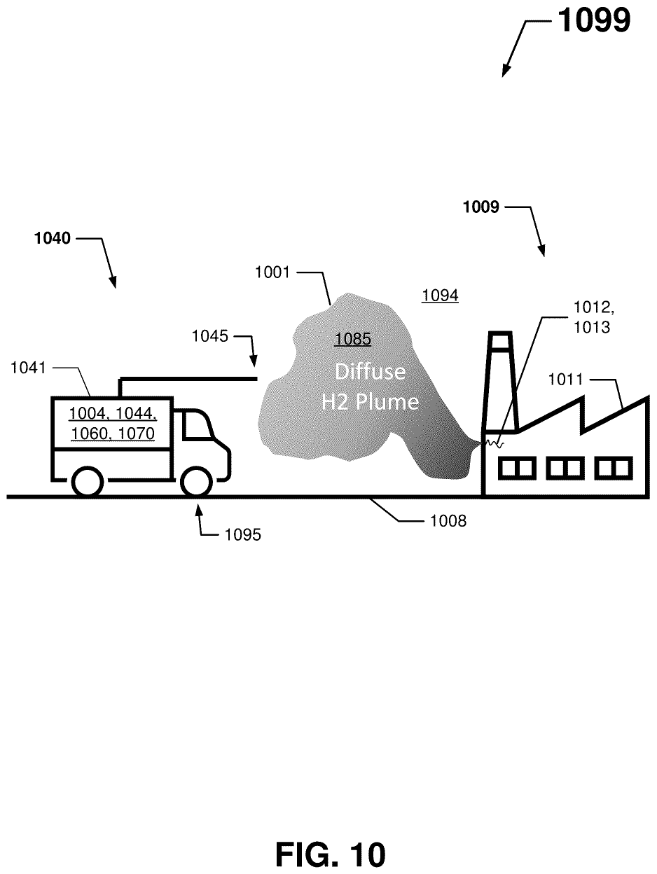

FIG. 10 shows a system 1099 that includes an example mobile vehicle 1040 according to certain example embodiments. Referring to the description above with respect to FIGS. 1 through 9, the system 1099 shows the mobile vehicle 1040 in the form of a truck. As a result, some of the mobility features 1095 of the mobile vehicle 1040 include tires and a combustion engine. In alternative embodiments, the combustion engine may be replaced with a battery-powered motor. The mobile vehicle 1040 is moving on the ground 1008 in an outside environment 1094 proximate to a storage vessel 1009 in the form of a H₂ generation plant.

In this case, examples of sensor devices 1060 on the mobile vehicle 1040 include a mix of CEAS, fluorescence, and absorption analyzers. The original samples (e.g., similar to original samples 471 discussed above) are drawn through individual sample lines to the sensor devices 1060 (e.g., analyzers) from sample collectors 1045 in the form of inlets that extend beyond the front edge of the mobile vehicle 1040 and approximately 3.3 m above the ground 1008. Positioning the sample collectors 1045 at this location relative to the body 1041 of the mobile vehicle 1040 largely eliminates exhaust contamination (except tailwinds).

By collecting and processing (using the processing apparatus 1044 of the mobile vehicle 1040) a large number of original samples (e.g., trace gasses), and subsequently measuring (using the one or more testing apparatuses 1070 of the mobile vehicle 1040) the resulting processed samples (e.g., similar to the processed samples 472 discussed above), provides the capability to fingerprint sources, including exhaust. Winds are measured by other sensor devices 1060 in the form of two 3D sonic anemometers positioned at approximately 3.3 m above the ground 1008 and approximately 50 cm in front of the body 1041 of the mobile vehicle 1040. Having duplicates of certain sensor devices 1060 (e.g., dual anemometers, dual GPSs) provide redundancy for certain measurements that may be critical in determining the existence and/or location of a leak point 1012 and/or failure 1013 in the field equipment 1011 (in this case, walls) of the storage vessel 1009 through which a gas 1085 escapes to form a leak 1001 (or plume) of the gas 1085 in the outside environment 1094. In certain example embodiments, accurate, true wind measurements by a sensor device 1060 requires correction (e.g., by a controller 1004 using one or more algorithms, one or more protocols, and/or stored data) for the velocity of the mobile vehicle 1040 and for anemometer-vehicle relative orientation, which may be measured by a 3-axis inclinometer on the anemometer.

Example embodiments may be used to provide systems and methods for detecting a gas leak in a storage vessel, including associated field equipment thereof, in an outside environment. In some cases, example embodiments also identify the leak source of the leak in the storage vessel, including associated field equipment thereof. In some cases, example embodiments further determine how to effectively reduce or stop the leak in the storage vessel, including associated field equipment thereof. In some cases, example embodiments also facilitate the repair of the leak in the storage vessel, including associated field equipment thereof. Example embodiments may provide a number of benefits. Such benefits may include, but are not limited to, ease of use, extending the life of a storage vessel (including associated field equipment thereof), flexibility, configurability, and compliance with applicable industry standards and regulations.

Although embodiments described herein are made with reference to example embodiments, it should be appreciated by those skilled in the art that various modifications are well within the scope of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

What is claimed is:

1. A method for detecting a leak of a gas from a storage vessel in an outside environment, the method comprising:
obtaining, by a sample collector on a mobile vehicle at a location in the outside environment, an original sample of the outside environment at the location;
processing, in real time by a processing apparatus on the mobile vehicle, the original sample to generate a processed sample by removing a naturally-occurring quantity of the gas from the original sample;
obtaining, from a sensor device on the mobile vehicle, a measurement of a parameter associated with the gas in the processed sample;
comparing, in real time using an analytic system on the mobile vehicle, the measurement to a range of acceptable values;
determining, in real time by the analytic system, that the measurement falls outside the range of acceptable values;
identifying, in real time based on the location and based on determining that the measurement falls outside the range of acceptable values, a characteristic of the leak in the storage vessel; and
reporting the leak in the storage vessel at the location in real time.

2. The method of claim 1, wherein the gas comprises at least one of a group consisting of dihydrogen and carbon dioxide.

3. The method of claim 1, wherein the storage vessel is subterranean.

4. The method of claim 3, wherein the storage vessel is defined by a subterranean formation.

5. The method of claim 3, wherein the storage vessel comprises a tank that is buried under ground.

6. The method of claim 1, wherein the mobile vehicle comprises at least one of a group consisting of a car, a truck, a trailer, and a van.

7. The method of claim 6, wherein the mobile vehicle is operated by a controller.

8. The method of claim 1, wherein the characteristic of the leak in the storage vessel comprises at least one of a group consisting of an extent of the leak, the location in the outside environment, a composition of the gas, the storage vessel from which the gas is leaking, a leak point of the storage vessel, and a failure in field equipment of the storage vessel.

9. The method of claim 1, further comprising:
obtaining, from a second sensor device on the mobile vehicle, a second measurement of a second parameter associated with the second gas in the processed sample;
comparing, in real time using the analytic system on the mobile vehicle, the second measurement to a second range of acceptable values; and
determining, in real time by the analytic system, that the second measurement falls outside the second range of acceptable values, wherein the source of the leak is further identified in real time based on the second measurement falling outside the second range of acceptable values.

10. The method of claim 1, further comprising:
obtaining, by the sample collector on the mobile vehicle at a second location in the outside environment, a second original sample of the outside environment at the second location;
processing, by the processing apparatus on the mobile vehicle, the second original sample to generate a second processed sample by removing the naturally-occurring quantity of the gas from the second original sample;
obtaining, from the sensor device on the mobile vehicle, a second measurement of the parameter associated with the gas in the second processed sample;
comparing, using the analytic system on the mobile vehicle, the second measurement to the range of acceptable values; and
determining, by the analytic system, that the second measurement falls outside the range of acceptable values,
wherein identifying the source of the leak in the storage vessel is further based on the second location.

11. The method of claim 10, wherein the second location is determined based on environmental conditions and based on determining that the measurement falls outside the range of acceptable values.

12. The method of claim 11, wherein the outside environmental conditions are measured by a second sensor device on the mobile vehicle.

13. The method of claim 1, wherein the location is determined by a global positioning system of the mobile vehicle.

14. The method of claim 1, further comprising:
evaluating how to repair the storage vessel to stop the leak; and
developing, based on evaluating how to repair the storage vessel, steps for a user to take to repair the storage vessel.

15. The method of claim 14, further comprising:
ordering equipment from a vendor, wherein the equipment is configured to be used to repair the storage vessel.

16. A mobile vehicle used to detect a leak of a gas from a storage vessel in an outside environment, the system comprising:
a sample collector configured to collect a plurality of original samples within the outside environment, wherein each of the plurality of original samples comprises the gas;
a processing apparatus configured to remove a naturally-occurring quantity of the gas from the plurality of samples to generate a plurality of processed samples;
a sensor device configured to measure an amount of the gas in each of the plurality of processed samples; and
an analytic system configured to:
compare, in real time, each measurement to a range of acceptable values:
determine, in real time, that at least one measurement falls outside the range of acceptable values; and
identify, in real time based and based on determining that the at least one measurement falls outside the range of acceptable values, a source of the leak in the storage vessel.

17. The mobile vehicle of claim 16, further comprising:
a mobility feature configured to move a body of the mobile vehicle within the outside environment, wherein the sample collector is mounted on the body, and wherein the processing apparatus, the sensor device, and the analytic system are at least partially disposed within the body.

18. The mobile vehicle of claim 17, wherein the mobility feature moves the body to an additional location within the outside environment after the analytic system determines that a measurement falls outside the range of acceptable values, wherein the additional location is determined by the analytic system.

19. The mobile vehicle of claim 16, further comprising:
a second sensor device configured to measure a parameter associated with the outside environment, wherein the parameter comprises at least one of a group consisting of a wind speed, a wind direction, a humidity, a temperature, and an atmospheric pressure.

20. A computer-implemented method for detecting a leak of a gas from a storage vessel in an outside environment using a mobile vehicle, the computer-implemented method comprising:
facilitate positioning the mobile vehicle at a location in the outside environment;
facilitate obtaining, by a sample collector on the mobile vehicle at the location in the outside environment, an original sample of the outside environment at the location;
facilitate processing, in real time by a processing apparatus on the mobile vehicle, the original sample to generate a processed sample by removing a naturally-occurring quantity of the gas from the original sample;
facilitate obtaining, from a sensor device on the mobile vehicle, a measurement of a parameter associated with the gas in the processed sample;
facilitate comparing, in real time using an analytic system on the mobile vehicle, the measurement to a range of acceptable values;
facilitate determining, in real time by the analytic system, that the measurement falls outside the range of acceptable values;
facilitate identifying, in real time based on the location and based on determining that the measurement falls outside the range of acceptable values, a characteristic of the leak in the storage vessel; and
facilitate reporting the leak in the storage vessel at the location in real time.
